SEQUENCE LISTING (5 sequences)

SEQ ID NO: 1 - RNA, 65 nt, Homo sapien
cuguuaaugcuaaucgugauagggguuuuugccuccaacugacuccuacauauuagcauuaacag

SEQ ID NO: 2 - RNA, 23 nt, Homo sapien
uuaaugcuaaucgugauaggggu

SEQ ID NO: 3 - RNA, 65 nt, Mus musculus
cuguuaaugcuaauugugauagggguuuuggccucugacugacuccuaccuguuagcauuaacag

SEQ ID NO: 4 - RNA, 23 nt, Mus musculus
uuaaugcuaauugugauaggggu

SEQ ID NO: 5 - RNA, 8 nt, Artificial Sequence
  oligonucleotide
uaaugcua

(19) United States
(12) Patent Application Publication
(10) Pub. No.: US 2012/0064122 A1
(43) Pub. Date: Mar. 15, 2012

(54) TREATMENT OF AUTOIMMUNE INFLAMMATION USING MIR-155

(76) Inventors: David Baltimore, Pasadena, CA (US); Ryan M. O'Connell, Pasadena, CA (US); Daniel Kahn, Calabasas, CA (US)

(21) Appl. No.: 13/230,695

(22) Filed: Sep. 12, 2011

Related U.S. Application Data
(60) Provisional application No. 61/382,426, filed on Sep. 13, 2010.

(51) Int. Cl. A61K 31/7088; A61P 37/04; A61P 31/00; A61P 31/04; A61P 31/12; C12N 5/0784; A61P 25/00; A61P 19/02; A61P 1/00; A61P 17/06; C12N 5/0783; A61P 29/00; A61P 37/00

(52) U.S. Cl. 424/278.1; 514/44 A; 435/375

(57) ABSTRACT

The present disclosure relates to the finding that microRNA-155 plays a role in the development and activity of CD4+ T cells. CD4+ T cell development and function, particularly T_{H}17 and T_{H}1 T cell development, can be modulated by delivery of microRNA-155 (miR-155) or antisense miR-155 to target CD4+ cells or precursor cells. In some embodiments, antisense miR-155 is used to reduce tissue specific autoimmune inflalmmation and to treat autoimmune disease. In addition, miR155 and antisense miR-155 can be used to modulate expression of cytokines from dendritic cells.

### CROSS-REFERENCE TO RELATED APPLICATIONS

The present application claims priority to U.S. Provisional application No. 61/382,426, filed Sep. 13, 2010, which is hereby incorporated by reference in its entirety.

### STATEMENT REGARDING FEDERALLY SPONSORED R&D

This invention was made with government support under AI079243, HL102228, CA133521, HD001400 awarded by the National Institutes of Health. The government has certain rights in the invention.

### REFERENCE TO SEQUENCE LISTING

The present application is being filed along with a Sequence Listing in electronic format. The Sequence Listing is provided as a file entitled SEQLISTING.TXT, created Sep. 12, 2011, which is 1.12 KB in size. The information in the electronic format of the Sequence Listing is incorporated herein by reference in its entirety.

### BACKGROUND OF THE INVENTION

1. Field of the Invention

The present application relates generally to activities of microRNA-155 and various uses of miR-155 arising therefrom.

2. Description of the Related Art

The mammalian inflammatory response has evolved to control infection by microbial pathogens before the onset of sepsis and death, while also playing important roles in tissue repair. Despite its utility, when the inflammatory response is activated inappropriately it may be directed against specific self-tissue antigens and cause serious disease. The outcome can be debilitating to important organ systems and is the underlying cause of many widespread human autoimmune disorders.

Recent work has revealed that IL-17 producing inflammatory CD4+ T cells, or T-helper (T_{H}) 17 cells, are mediators of chronic, autoimmune inflammation. T_{H}17 development is driven by cytokines produced primarily by cells of the innate immune system, including TGF-β, IL-6, IL-23 and IL-1. Overexpression of IL-17 has been shown to lead to increased granulopoiesis in vivo (Schwarzenberger et al. (1998). IL-17 stimulates granulopoiesis in mice: use of an alternate, novel gene therapy-derived method for in vivo evaluation of cytokines. J Immunol 161, 6383-6389), and inhibition of IL-17 in mice can ameliorate several autoimmune disorders including experimental autoimmune encephalomyelitis (EAE), collagen induced arthritis (CIA), and inflammatory bowel disease (IBD) (Ivanov et al. (2006). The orphan nuclear receptor RORgammat directs the differentiation program of proinflammatory IL-17+ T helper cells. Cell 126, 1121-1133; Komiyama et al. (2006). IL-17 plays an important role in the development of experimental autoimmune encephalomyelitis. J Immunol 177, 566-573; Murphy et al. (2003). Divergent pro- and antiinflammatory roles for IL-23 and IL-12 in joint autoimmune inflammation. J Exp Med 198, 1951-1957).

miRNAs (miR) are a novel class of non-coding RNAs that modulate gene expression at the posttranscriptional level. A number of miRNAs have been found to be involved in regulating several aspects of inflammation: miR-155a, miR-155 and miR-132, were initially shown to be upregulated during the macrophage inflammatory response (O'Connell et al. (2007). MicroRNA-155 is induced during the macrophage inflammatory response. Proc Natl Acad Sci USA 104, 1604-1609; Taganov et al. (2006). NF-kappaB-dependent induction of microRNA miR-146, an inhibitor targeted to signaling proteins of innate immune responses. Proc Natl Acad Sci USA 103, 12481-12486), and several more have since been reported including miR-147, miR-9, let-7e and miR-21 (Androulidaki et al. (2009). The kinase Akt1 controls macrophage response to lipopolysaccharide by regulating microRNAs. Immunity 31, 220-231; Bazzoni et al. (2009). Induction and regulatory function of miR-9 in human monocytes and neutrophils exposed to proinflammatory signals. Proc Natl Acad Sci USA 106, 5282-5287; Liu et al. (2009). miR-147, a microRNA that is induced upon Toll-like receptor stimulation, regulates murine macrophage inflammatory responses. Proc Natl Acad Sci USA 106, 15819-15824; Sheedy et al. (2009). Negative regulation of TLR4 via targeting of the proinflammatory tumor suppressor PDCD4 by the microRNA miR-21. Nat. Immunol.).

miR-155 was among the first miRNAs linked to some types of inflammation by virtue of its potent upregulation in some immune cell lineages by Toll Like Receptor (TLR) ligands, inflammatory cytokines, and specific antigens (Haasch et al. (2002). T cell activation induces a noncoding RNA transcript sensitive to inhibition by immunosuppressant drugs and encoded by the proto-oncogene, BIC. Cell Immunol 217, 78-86; O'Connell et al. (2007). MicroRNA-155 is induced during the macrophage inflammatory response. Proc Natl Acad Sci USA 104, 1604-1609; Taganov et al. (2006). NF-kappaB-dependent induction of microRNA miR-146, an inhibitor targeted to signaling proteins of innate immune responses. Proc Natl Acad Sci USA 103, 12481-12486; That et al. (2007). Regulation of the germinal center response by microRNA-155. Science 316, 604-608). A wide variety of immunologically relevant targets of miR-155 have been reported, indicating distinct roles in mammalian immunity. Among these roles, miR-155 has been shown to be important for immunoglobulin class switching to IgG in B cells via targeted repression of AID and PU.1 (Dorsett et al. (2008). MicroRNA-155 suppresses activation-induced cytidine deaminase-mediated Myc-Igh translocation. Immunity 28, 630-638; Rodriguez et al. (2007). Requirement of bic/microRNA-155 for normal immune function. Science 316, 608-611; That et al. (2007). Regulation of the germinal center response by microRNA-155. Science 316, 604-608; Vigorito et al. (2007). microRNA-155 regulates the generation of immunoglobulin class-switched plasma cells. Immunity 27, 847-859). The fitness of T regulatory cells (Tregs) is influenced by targeted repression of SOCS1 by miR-155 (Kohlhaas et al. (2009). Cutting edge: the Foxp3 target miR-155 contributes to the development of regulatory T cells. J Immunol 182, 2578-2582; Lu et al. (2009). Foxp3-dependent microRNA155 confers competitive fitness to regulatory T cells by targeting SOCS1 protein. Immunity 30, 80-91). In myeloid cells, overexpression of miR-155 drives a myeloproliferative disorder through a mechanism involving repression of SHIP1 (O'Connell et al. (2009). Inositol phosphatase SHIP1 is a primary target of miR-155. Proc Natl Acad Sci USA 106, 7113-7118; O'Connell et al. (2008). Sustained expression of microRNA-155 in hematopoietic stem cells causes a myeloproliferative disorder. J Exp Med 205, 585-594).

### SUMMARY OF THE INVENTION

In one aspect, methods or reducing tissue specific autoimmune inflammation in a subject are provided. A subject in need of a reduction in autoimmune inflammation may be identified and administered an anti-miR-155, such as an antisense miR-155 oligonucleotide. A reduction in autoimmune inflammation may be measured.

In some embodiments, the anti-miR-155 is delivered to a population of CD4+ T cells, for example in an inflamed tissue. Delivery may be, for example, by contacting the tissue with an expression vector encoding an anti-miR-155 oligonucleotide, or by contacting the tissue with an anti-miR-155 oligonucleotide directly.

In some embodiments an antisense miR-155 oligonucleotide comprises a nucleic acid sequence that is complementary to a miR-155 nucleic acid selected from SEQ IN NOs: 1-5.

In another aspect, methods of decreasing CD4+ T cell proliferation are provided. The methods may comprise administering an anti-miR-155, such as an antisense miR-155 oligonucleotide, to a population of cells comprising CD4+ T cells. Proliferation of the CD4+ T cells may be measured after administration of the anti-miR-155. The CD4+ T cells may be, for example, T_{H}1 and/or T_{H}17 cells.

In another aspect, methods of decreasing cytokine production by dendritic cells are provided, in which miR-155 activity is inhibited in the dendritic cells. In some embodiments the cytokines are selected from the group consisting of IL-23/IL-17, GM-CSF, IL-6, IFNγ and TNF-α. miR-155 activity can be inhibited, for example, by administering an antisense miR-155 oligonucleotide to the dendritic cells.

In another aspect, methods of increasing an immune response to an infectious agent in a subject are provided. A subject suffering from infection is identified and an anti-miR-155 is administered. The anti-miR-155 may be, for example, an antisense miR-155 oligonucleotide. The anti-miR-155 may be administered systemically or specifically to an area of infection, such as an infected tissue. The immune response may be measured before and/or after administration of the anti-miR-155. In some embodiments, the immune response may be measured by measuring proliferation of CD4+ T cells in the subject, such as T_{H}1 and/or T_{H}17 T cells.

In another aspect, methods of treating autoimmune disorders are provided. The autoimmune disorders that may be treated include, for example, multiple sclerosis, rheumatoid arthritis, lupus, irritable bowel syndrome and psoriasis. In some embodiments a patient suffering from tissue specific autoimmune inflammation is identified and treated. The patient may be administered an anti-miR-155, such as an antisense miR-155 oligonucleotide. In some embodiments a reduction in tissue specific autoimmune inflammation is measured after administration of the anti-miR-155.

In another aspect, methods of reducing development of T_{H}1 and T_{H}17 cells in a tissue undergoing autoimmune inflammation are provided. The methods may comprise identifying a tissue undergoing autoimmune inflammation and administering an anti miR-155, such as an antisense miR-155 oligonucleotide to the tissue. In some embodiments administering the antisense miR-155 oligonucleotide to the tissue comprises contacting the tissue with an expression vector encoding an antisense miR-155 oligonucleotide. Development of T_{H}1 and T_{H}17 cells in the tissue can be measured.

### BRIEF DESCRIPTION OF THE DRAWINGS

FIG. 1. miR-155−/− mice are resistant to EAE induced by MOG₃₅₋₅₅. A. Disease severity is illustrated, based upon clinical symptoms (n=10). B. Disease incidence is illustrated, as assessed for each group (n=10). C. Representative H&E stained brain sections from miR-155+/+ or miR-155−/− mice harvested on day 25 post-immunization are shown. D. The average histology score for each group (n=4) is illustrated. E. The number of live DLN cells (left) and their lineage composition is shown, as assessed by FACS (right) using DLNs from miR-155+/+ and miR-155−/− mice 25 days post immunization (n=4). F. The number of splenocytes (left) and their lineage composition is shown, as determined by FACS (right) using spleens from both groups 25 days post immunization (n=4). G. Expression of miR-155 in LPS activated splenic B cells is illustrated. H. MOG₃₅₋₅₅-induced EAE disease severity is illustrated, as scored over a time course (n=5-7). I. Disease severity following adoptive transfer was scored and is shown over time. Error bars represent +/−SEM and * denotes statistical significance with a p value of <0.05 according to a student's two-tailed t-test.

FIG. 2. miR-155−/− mice exhibit defective inflammatory T cell development during EAE. A. Intracellular staining identified total lymph node cells (top) and CD4+ lymphocytes (bottom) producing IL-17A and/or IFNγ. The average of 4 mice per group is shown on the right. B. Splenocytes were analyzed as in (A). C. CFSE loss by CD4+ proliferating cells from both groups is illustrated, as assayed by FACS following restimulation with MOG₃₅₋₅₅ (20 μg/ml) for 72 hours. D. Splenocyte recall response to MOG₃₅₋₅₅ is illustrated, as assessed by ³[H] thymidine incorporation. E. Production of IL-17A and IFNγ by cells from (C.) is illustrated, as determined by ELISA. Error bars represent +/−SEM and * denotes statistical significance with a p value of <0.05 according to a student's two-tailed t-test.

FIG. 3. miR-155 is required for inflammatory T cell development during the induction phase of EAE. A. CD4+ lymphocytes producing IL-17A and/or IFNγ are shown. B. Total numbers of T_{H}17 and T_{H}1 cells in the brain are illustrated, in addition to the percentage of Th17 and Th1 cells among total CD4+ T cells. C. Expression of BIC, IL-17A and IL-23 p19 mRNA in wt and miR-155−/− splenocytes is illustrated. D. The number of T_{H}17 and T_{H}1 cells is shown. E. Illustrates the splenocyte recall response following stimulation with MOG₃₅₋₅₅ and F. Illustrates the production of IL-17A, IFNγ, IL-6 and GM-CSF as measured by ELISA. G. Expression of BIC and IL-17A mRNA in the DLNs is illustrated and H. the number of T_{H}17 and T_{H}1 cells is shown, as quantified by FACS.

FIG. 4. miR-155−/− mice have reduced foot pad inflammation during DTH. miR-155+/+ and miR-155−/− mice were immunized with 100 μg of KLH in CFA and 8 days later injected with 50 μg of KLH in one footpad and PBS in the other (n=5). A. Increases in footpad inflammation are shown for both groups. B. Total numbers of splenocytes and DLN cells are shown. C. Proliferation of splenocytes and DLN cells following in vitro restimulation with KLH is illustrated, as determined by assaying ³[H] thymidine incorporation. D. Production of IL-17A, IFNγ and IL-6 from the cells in (C.) is illustrated, as determined by ELISA. Error bars represent +/−SEM and * denotes statistical significance with a p value of <0.05 according to a student's t-test.

FIG. 5. miR-155 expression by CD4+ T cells is necessary for proper T_{H}17 development in vitro. CD4+ T cells were isolated from miR-155+/+ or miR-155−/− spleens and cultured in the presence of plate bound αCD3 and soluble αCD28 antibodies, with (T_{H}17) and without (T_{H}0) IL-6 (50 ng/ml) and TGFβ (2 ng/ml). A. After 96 hours, expression of IL-17A and IFNγ was assayed by intracellular staining following by FACS, as illustrated. B. Results from a representative experiment are represented graphically. The experiment was performed 3 independent times. C. Expression of miR-155 is shown, as measured by qPCR before and after activation with αCD3/CD28 antibodies. D. Expression of BIC, miR-155, and IL-17A in Wt and miR-155−/− CD4+ T cells is shown, as assayed by qPCR following 96 hours of culture with αCD3/CD28 antibodies alone, or in T_{H}17 skewing conditions. Error bars represent +/−SEM.

FIG. 6. Expression of miR-155 by CD4+ T cells is required for proper development of inflammatory T cells during EAE. A. 5×10⁶ Wt or miR-155−/− CD4+ T cells from naïve mice were injected i.v. into Rag1−/− recipients. Development of EAE induced with MOG₃₅₋₅₅ 24 hours later is illustrated over a time course (n=5-6). B. Mice were harvested and engraftment of CD3+ CD4+ T cells is shown, as assayed by FACS using splenocytes (top). Expression of IL-17A and IFNγ by CD4+ cells in the spleens and DLNs was assayed by intracellular staining following by FACS. A representative plot from the DLNs is shown (bottom). C. Data from 5-6 mice are shown graphically. D. miR-155+/+ and miR-155−/− mice were injected with 10⁷ Wt CD45.1+ CD4+ naïve T cells, and EAE was induced 24 hours later (n=5). Disease symptoms are shown over a time course. E. Mice were harvested and CD4+ T cells in the brains were analyzed by FACS to detect cells expressing CD45.1, IL-17A and IFNγ. F. Data from 5 mice per group are represented graphically. Error bars represent +/−SEM and * denotes statistical significance with a p value of <0.05 according to a student's two-tailed t-test.

FIG. 7. miR-155 expression in LPS-activated, GM-CSF-derived myeloid dendritic cells is necessary for proper production of T_{H}17 relevant inflammatory cytokines. A. CD11c+DCs were derived using recombinant mouse GM-CSF at 20 ng/ml, as shown. B. Expression of BIC (top) and mature miR-155 (bottom) before and after 20 hours of LPS stimulation (100 ng/ml) was assayed using qPCR. C. mRNA expression differences between miR-155+/+ and miR-155−/− LPS treated DCs is illustrated. Several selected targets of miR-155 were expressed higher in miR-155−/− DCs, while a subset of selected proinflammatory cytokines were expressed lower. D. Expression of SHIP1 and SOCS1 mRNAs was assessed by qPCR and by Western blotting (n=3). E. qPCR was used to assay expression of IL-23 p19, IL-6, IL-12p40 and TNFα mRNA levels (n=3), as illustrated. F. Concentrations of the cytokines from (E) in the culture supernatants are shown, as determined by ELISAs (n=3). G. GM-CSF-derived DCs overexpressing miR-155, a miR-155 seed mutant or control vector were stimulated with LPS for 20 hours and expression of IL-23 p19, IL-6, IL-12p40 and TNFα mRNA is illustrated, as assayed by qPCR. H. Proliferation of 2D2 or OT2 CD4+ T cells in response to their respective antigens presented by Wt or miR-155−/− DCs is shown, as assessed by assaying ³[H] thymidine incorporation. Error bars represent +/−SEM and * denotes statistical significance with a p value of <0.05 according to a student's two-tailed t-test.

FIG. 8. The disease incidence of Wt or miR-155−/− mice is illustrated after receiving day 12 Wt encephalitogenic splenocytes.

FIG. 9. The number of IL-17A or IFNγ positive CD4+ T cells in Wt and miR-155−/− spleens and DLNs from day 24 EAE mice is shown.

FIG. 10. The total number of live cells from the spleens, DLNs or brains of wt or miR-155−/− day 13 EAE mice is illustrated.

FIG. 11. A. the percentage and absolute numbers of FoxP3+ CD4+ Treg cells in the lymph notes and spleens of miR-155+/+ or −/− mice with EAE is shown as determined 25 days post immunization with MOG₃₅₋₅₅. B. and C. The averages of 4 mice in each group are shown.

FIG. 12. Relative levels of MOG₃₅₋₅₅ reactive IgG antibodies in the serum of miR-155+/+ and −/− mice, as determined gy FACS 25 days after immunization with MOG₃₅₋₅₅.

### DETAILED DESCRIPTION OF THE PREFERRED EMBODIMENT

The micro-RNA miR-155 plays an important role in driving chronic inflammation that is inappropriately directed at tissue specific antigens, a destructive process that is at the heart of human autoimmune diseases. At the cellular level, mice lacking miR-155 (miR-155−/− mice) exhibit defective inflammatory T cell development during the induction phase of autoimmunity. In addition miR-155 enhances production of certain cytokines by inflammatory dendritic cells (DCs).

Antisense miR-155 or other miR-155 antagonist can be delivered to target T cells to reduce or prevent inflammatory T cell development. In some embodiments, miR-155 antagonists, such as antisense miR-155 are used to treat chronic inflammation. For example, miR-155 antagonists can be delivered to T cells, particularly CD4+ T cells. In some embodiments, patients suffering from an autoimmune disorder, such as multiple sclerosis (MS), rheumatoid arthritis (RA), inflammatory bowel disease, psoriasis and Systemic Lupus Erythematosus (SLE), are treated by administering a miR-155 antagonist, such as antisense miR-155 to target cells such as CD4+ T cells.

miR-155 can be used to enhance an immune response to an infection. For example, in patients suffering from or expected to suffer from infections, miR-155 can be used to increase the immune response to the infectious agent. In some embodiments miR-155 is delivered to target cells such as CD4+ T cells.

In some embodiments, antisense miR-155, or other miR-155 antagonist, is used to reduce production of one or more cytokines selected from IL-23/IL-17, GM-cCSF, IL-6, IFNγ and TNF-α by dendritic cells. The dendritic cells may be contacted with the antisense miR-155 or other antagonist In some embodiments production of two or more, three, four or all five of these cytokines is reduced.

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present invention, the following terms are defined below.

When used herein the terms “miR,” “mir” and “miRNA” are used to refer to microRNA, a class of small RNA molecules that are capable of modulating RNA translation (see, Zeng and Cullen, RNA, 9(1):112-123, 2003; Kidner and Martienssen Trends Genet, 19(1):13-6, 2003; Dennis C, Nature, 420(6917):732, 2002; Couzin J, Science 298(5602):2296-7, 2002, each of which is incorporated by reference herein).

The terms “miR,” “mir” and “miRNA,” unless otherwise indicated, include the mature, pri-, pre-form of a particular microRNA as well as the seed sequence of the microRNA, sequences comprising the seed sequence, and variants thereof. For example, the terms “MiRNA-155” and “miR-155” are used interchangeably and, unless otherwise indicated, refer to microRNA-155, including miR-155, pri-miR-155, pre-miR-155, mature miR-155, miRNA-155 seed sequence, sequences comprising a miRNA-155 seed sequence, and any variants thereof miR-155 sequences are known in the art. Exemplary sequences include mature human miR-155 (SEQ ID NO: 1), human pre-miR-155 (SEQ ID NO: 2), murine miR-155 (SEQ ID NO: 3), murine pre-miR-155 (SEQ ID NO: 4) and miR-155 seed sequences (SEQ ID NO: 5), and variants thereof. Other miR-155 sequences are known in the art, for example as disclosed in Eis et al. (2005) Proc. Nat. Acad. Sci. USA 102:3627-3632, which is incorporated by reference herein.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, such as constitutive or inducible promoters.

“MiRNA nucleic acid” is defined as RNA or DNA that encodes a miR as defined above, or is complementary to a nucleic acid sequence encoding a miR, or hybridizes to such RNA or DNA and remains stably bound to it under appropriate stringency conditions. Specifically included are genomic DNA, cDNA, mRNA, miRNA and antisense molecules, pri-miRNA, pre-miRNA, mature miRNA, miRNA seed sequence, as well as nucleic acids based on alternative backbones or including alternative bases. MiRNA nucleic acids can be derived from natural sources or synthesized.

“MicroRNA seed sequence,” “miRNA seed sequence,” “seed region” and “seed portion” typically refer to nucleotides 2-7 or 2-8 of the mature miRNA sequence. The miRNA seed sequence is typically located at the 5′ end of the miRNA. An exemplary miR-155 seed sequences are provided in SEQ ID NOs: 5. Other miR-155 seed sequences will be apparent to the skilled artisan.

“miR antagonist” means an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleobase sequence that is complementary to the nucleobase sequence of a miRNA, or a precursor thereof. In certain embodiments, a miR antagonist is a miR-155 antagonist. In other embodiments, an miR-155 antagonist comprises a small molecule, or the like that interferes with or inhibits the activity of an miRNA.

“miR-155 antagonist” and “anti-miR-155” means an agent designed to interfere with or inhibit the activity of miR-155. In some embodiments an anti-miR-155 is a miR-155 antisense molecule.

The term “operably linked” is used to describe the connection between regulatory elements and a gene or its coding region. That is, gene expression is typically placed under the control of certain regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element.

As used herein, the term “variant” refers to a polynucleotide having a sequence substantially similar to a reference polynucleotide. A variant can comprises deletions, substitutions, additions of one or more nucleotides at the 5′ end, 3′ end, and/or one or more internal sites in comparison to the reference polynucleotide. Similarities and/or differences in sequences between variants and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variants of a particular polynucleotide disclosed herein, including, but not limited to, a miRNA, will have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans.

The term “mammal” is defined as an individual belonging to the class Mammalia and includes, without limitation, humans, domestic and farm animals, and zoo, sports, or pet animals, such as sheep, dogs, horses, cats or cows. Preferably, the mammal herein is human. However, in some embodiments the mammal is not a human.

As used herein, “treatment” is a clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Those in need of treatment include those already affected by a disease or disorder or undesired physiological condition as well as those in which the disease or disorder or undesired physiological condition is to be prevented.

The term “effective amount” refers to an amount sufficient to effect beneficial or desirable biological and/or clinical results.

“Pharmaceutically acceptable” carriers, excipients, or stabilizers are ones which are nontoxic to the cell or mammal being exposed thereto at the dosages and concentrations employed or that have an acceptable level of toxicity as determined by the skilled practitioner. Often the physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™.

### MiR-155 Nucleic Acid Molecules

Nucleic acid molecules that encode miR-155 are used in various embodiments. miR-155 sequences for mature miR-155, pre-miR-155 and miR-155 seed sequence are provided in SEQ ID NOs: 1-5 and are used in some embodiments. A miRNA sequence may comprise from about 6 to about 99 or more nucleotides. In some embodiments, a miRNA sequence comprises about the first 6 to about the first 22 nucleotides of a pre-miRNA-155, or about the first 10 to about the first 20 nucleotides of a pre-miRNA-155. In some embodiments, the miRNA can be an isolated or purified oligonucleotide of at least 6, 7, 8, 10, 11, 12, 13, 14, 15, 16, 17, 18, 19, 20, 21, 22, 23, 24, or 25 nucleotides in length. In some embodiments, the miRNA is a hybridizable portion of a miR-155 sequence or its complementary sequence.

Isolated or purified polynucleotides having at least 6 nucleotides (i.e., a hybridizable portion) of a miR-155 coding sequence or its complement are used in some embodiments. In other embodiments, miR-155 polynucleotides preferably comprise at least 22 (continuous) nucleotides, or a full-length miR-155 coding sequence.

In some embodiments, nucleic acids are used that are capable of blocking the activity of miRNA-155 (anti-miRNA 155 or anti-miR-155). Such nucleic acids may include, for example, antisense miR-155 oligonucleotides. In some embodiments, the anti-miR-155 is an antisense miR-155 nucleic acid comprising a total of about 5 to about 100 or more, more preferably about 10 to about 60 nucleotides. In some embodiments, an anti-miRNA may comprise a total of at least about 5 to about 26 nucleotides. In some embodiments, the sequence of the anti-miRNA can comprise at least 5 nucleotides that are substantially complementary to the 5′ region of a miR-155, at least 5 nucleotides that are substantially complementary to the 3′ region of a miR-155, at least 4-7 nucleotides that are substantially complementary to a miR-155 seed sequence, or at least 5-12 nucleotide that are substantially complementary to the flanking regions of a miR-155 seed sequence.

In some embodiments, an anti-miR-155 is a nucleic acid that comprises the complement of a sequence of a miR-155 referred to in SEQ ID NOs: 1-5. In some embodiments an anti-miR-155 is an antisense oligonucleotide that is able to hybridize to a miR-155 of SEQ ID NOs: 1-6. In some embodiments the antisense miR-155 oligonucleotide is able to hybridize under stringent conditions to a nucleic acid comprising a sequence of SEQ ID NOs: 1-6.

In some embodiments, a miR-155 antisense oligonucleotide has a sequence that is complementary to a sequence having at least 80%, at least 85%, at least 90%, at least 95%, or at least 98% identity to the miR-155 sequence set forth in SEQ ID NO: 1 or 3. In some embodiments, a miR-155 antisense oligonucleotide is able to hybridize, for example under stringent conditions, to a nucleic acid comprising a sequence having at least 80%, at least 85%, at least 90%, at least 95%, or at least 98% identity to the miR-155 sequence set forth in SEQ ID NO: 1 or 2.

In some embodiments, a miR-155 antisense oligonucleotide has a sequence that is complementary to a sequence having at least 80%, at least 85%, at least 90%, at least 95% or at least 98% identity to a pre-miR-155 sequence as in SEQ ID NO: 2 or 4. In some embodiments, a miR-155 antisense oligonucleotide is able to hybridize, for example under stringent conditions, to a nucleic acid comprising a sequence having at least 80%, at least 85%, at least 90%, at least 95% or at least 98% identity to a pre-miR-155 sequence as in SEQ ID NO: 2 or 4.

In some embodiments, a miR-155 antisense oligonucleotide has a sequence that is complementary to a sequence having at least 80%, at least 85%, at least 90%, at least 95%, or at least 98% identity to a sequence of a miR-155 seed sequence, such as a seed sequence set forth in SEQ ID NOs:5. In some embodiments, a miR-155 antisense oligonucleotide is able to hybridize to a nucleic acid comprising sequence having at least 80%, at least 85%, at least 90%, at least 95%, or at least 98% identity to a sequence of a miR-155 seed sequence, such as a seed sequence set forth in SEQ ID NOs:5.

In some embodiments, anti-miR-155 molecules are those that are able to hybridize under stringent conditions to the complement of a cDNA encoding a miR-155, for example a cDNA encoding a miR-155 comprising the sequence of any of SEQ ID NOs: 1-5.

It is not intended that the methods be limited by the source of the miR-155 or anti-miR-155. The miR-155 and/or anti-miR-155 can be from a human or non-human mammal, derived from any recombinant source, synthesized in vitro or by chemical synthesis. The nucleic acid may be DNA or RNA and may exist in a double-stranded, single-stranded or partially double-stranded form, depending on the particular context. miR-155 and anti-miR-155 nucleic acids may be prepared by any conventional means typically used to prepare nucleic acids. For example, nucleic acids may be chemically synthesized using commercially available reagents and synthesizers by methods that are well-known in the art for example, the phosphotriester method of Matteucci, et al., (J. Am. Chem. Soc. 103:3185-3191, 1981) and/or using automated synthesis methods. (See, e.g., Gait, 1985, Oligonucleotide Synthesis: A Practical Approach, IRL Press, Oxford, England). In addition, DNA or RNA segments can readily be prepared by well known methods, such as synthesis of a group of oligonucleotides that define various modular segments, followed by ligation of oligonucleotides to build the complete segment. Unless otherwise indicated, the various embodiments are not limited to naturally occurring miR-155 sequences; mutants and variants of miR-155 sequences may also be used.

In some embodiments, a synthetic miRNA can have a sequence that is different from a naturally-occurring miRNA-155 and effectively mimic the naturally-occurring miRNA. For example, the synthetic miRNA can have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to the naturally-occurring miRNA. In other embodiments the synthetic miRNA can have a sequence that is different from the complement of a naturally-occurring miR-155. For example, the synthetic miRNA can have at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to the complement of a naturally-occurring miRNA.

Nucleotide sequences that encode a mutant of a miR-155, such as a miR-155 with one or more substitutions, additions and/or deletions, and fragments of miR-155 as well as truncated versions of miR-155 maybe also be useful in some of the methods disclosed herein.

Modified nucleotides or backbone modifications can be utilized in some embodiments, for example to increase stability and/or optimize delivery of sense or antisense oligonucleotides. For example, modified nucleotides may include: linked nuclear acid (LNA), 2′-O-Me nucleotides, 2′-O-methoxyethyl, and 2′ fluoro. Backbone modifications include, for example, phosphorothioate and phosphate.

In some embodiments, a miR-155 or anti-miR-155 oligonucleotide is modified with cholesterol to enhance delivery to target cells. The cholesterol can be linked, for example, through a hydroxyprolinol linkage on the 3′ end of the miRNA.

Nucleic acid molecules encoding miR-155 and anti-miR-155 are used in some embodiments, for example to modulate function, activity and/or proliferation of immune cells, particularly CD4+ T cells and/or dendritic cells. Anti-miR-155 can be used, for example to treat autoimmune disorders and miR-155 can be used, for example, to enhance the immune response to some infections.

### Inhibition of Micro-RNAs

The present disclosure provides inhibitors of miR-155 (i.e., anti-miR-155). Compositions comprising such inhibitors and methods for inhibiting miR-155 using such inhibitors are also disclosed herein. Any miRNA inhibitor may be used alone, or with other miRNA inhibitor(s) known in the art. In some embodiments, the miRNA inhibitor is a nucleic acid-based inhibitor that is capable of forming a duplex with the target miRNA by Watson-Crick type base pairing. One of the non-limiting examples of the nucleic acid-based miRNA inhibitor is an antisense oligonucleotide. It is not necessary that there be perfect complementarity between the nucleic acid-based miRNA inhibitor and the target miR-155. The miRNA inhibitor may have one or more regions of non-complementarity with the target miR-155 flanked by one or more regions of complementarity sufficient to allow duplex formation. In some embodiments, the regions of complementarity can be at least 6, 7, 8, 9, 10, 11, 12, 13, 14, 15, 16, 17, 18, 19, 20, 21, 22, 23, 24, or 25 nucleotides long.

As used herein, the mechanism by which the miRNA inhibitor functions to inhibit the activity of the target miR-155 is not limited in any way. For example, a nucleic acid-based inhibitor, in some embodiments, may form a duplex with the target miR-155 sequences and prevent proper processing of the mature miR-155 product from its precursor, or may prevent the mature miR-155 from binding to its target gene, or may lead to degradation of pr-, pre-, or mature miR-155, or may act through some other mechanism.

In some embodiments, a miR-155 inhibitor is used to attenuate, reduce, block, or abolish the activity of the target miR-155. The extent to which the activity of the miR-155 is reduced can vary. For example, the miR-155 inhibitors disclosed herein can reduce the activity of the target miR-155 by at least about 10%, at least about 20%, at least about 30%, at least about 40%, at least about 50%, at least about 60%, at least about 70%, at least about 80%, at least about 90%, at least about 95%, at least about 98%, or at least about 99%. In some embodiments, the miR-155 inhibitor can completely abolish the activity of the target miR-155. Non-limiting examples of miRNA inhibitors include nucleic acids that can block the activity of a miRNA, such as an antisense miRNA. Such nucleic acids include, for example, antisense miR-155 oligonucleotides. In some embodiments, the anti-miRNA can have a total of at least about 5 to about 26 nucleotides. In some embodiments, the sequence of the anti-miRNA can have at least 5, 6, 7, 8, 9, 10, 11, 12, 13, 14, 15, 16, 17, 18, 19, 20, or 21 nucleotides that are substantially complementary to the 5′ region of a miR-155; or at least 5, 6, 7, 8, 9, 10, 11, 12, 13, 14, 15, 16, 17, 18, 19, 20, or 21 nucleotides that are substantially complementary to the 3′ region of a miR-155. In some embodiments, the sequence of the anti-miRNA can comprise at least 4-7 nucleotides that are substantially complementary to a miR-155 seed sequence. In some embodiments, the sequence of the anti-miRNA can comprise at least 5-12 nucleotide that are substantially complementary to the flanking regions of a miR-155 seed sequence. In some embodiments, the anti-miRNA is an antisense miR-155 nucleic acid comprising a total of about 5 to about 100 or more nucleotides, more preferably about 10 to about 60 nucleotides or about 15 to about 30 nucleotides, and has a sequence that is preferably complementary to at least the seed region of miR-155. It has been shown that antisense miRNAs can specifically silence target miRNA in tissue. Krutzfeldt, J. et al., Nature, 438:685-9 (2005).

As disclosed herein, the anti-miR-155, for example an antisense miR-155 oligonucleotide, can be from a human or non-human mammal, derived from any recombinant source, synthesized in vitro or by chemical synthesis. Oligonucleotides can be DNA or RNA, and can in a double-stranded, single-stranded or partially double-stranded form. The oligonucleotides can be prepared by any conventional means known in the art to prepare nucleic acids. For example, nucleic acids may be chemically synthesized using commercially available reagents and synthesizers by methods that are well-known in the art, including, but not limited to, the phosphotriester method described in Matteucci, et al., (J. Am. Chem. Soc. 103:3185-3191, 1981) and/or an automated synthesis method described in Gait (Oligonucleotide Synthesis: A Practical Approach, 1985, IRL Press, Oxford, England). Larger DNA or RNA segments can also readily be prepared by conventional methods known in the art, such as synthesis of a group of oligonucleotides that define various modular segments, followed by ligation of oligonucleotides to build the complete segment.

The miRNA inhibitors can comprise modified or unmodified nucleotides. In some embodiments, modified nucleotides or backbone modifications can be used to increase stability and/or optimize delivery of the sense or antisense oligonucleotides. Non-limiting modified nucleotides include linked nuclear acid (LNA), 2′-O-Me nucleotides, 2′-O -methoxyethyl, and 2′ fluoro. Backbone modifications include, but are not limited to, phosphorothioate and phosphate. In some embodiments, a microR-155 or an antisense miR-155 oligonucleotide disclosed herein can be modified with cholesterol to enhance delivery to target cells. The cholesterol can be linked, for example, through a hydroxyprolinol linkage on the 3′ end of the microRNA.

In some embodiments, the miRNA inhibitor can comprise ribonucleotides, deoxyribonucleotides, 2′-modified nucleotides, phosphorothioate-linked deoxyribonucleotides, peptide nucleic acids (PNAs), locked nucleic acids (LNAs), or other forms of naturally or non-naturally occurring nucleotides. The miRNA inhibitor can comprise nucleobase modifications, include, but not limited to, 2-amino-A, 2-thio (e.g., 2-thio-U), G-clamp modifications, antagomirs, morpholinos, nucleic acid aptamers, or any other type of modified nucleotide or nucleotide derivative that is capable of Watson-Crick type base pairing with a miRNA. As an example, in addition to naturally occurring DNA and/or RNA nucleotide bases, non-naturally occurring modified nucleotide bases that can be used in the miRNA inhibitors disclose herein, include, but are not limited to, 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carb 1 pseudouridine, beta-D-galactosylqueosine, 2′-Omethylguanosine, inosine, N⁶-isopentenyladenosine, 1-methyladenosine, 1-methylpseudouridine, 1-methylguanosine, 1-methylaminomethyllinosine, 2,2-dimethylguanosine, 2-methyladenosine, 2-methylguanosine, 3-methylcytidine, 5-methylcytidine, N.sup.6-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N-6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid methylester uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl)carbamoyl)threonine, 2′-O-methyl-5-methyluridine, 2′-O-methyluridine, wybutosine, and 3-(3-amino-3-carboxypropyl)uridine. In some embodiments, the miRNA inhibitor comprises morpholinos or antagomirs.

The miRNA inhibitors disclosed herein can be further modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. A 3′ conjugate, such as naproxen or ibuprofen, may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

The 5′-terminus of the miRNA inhibitors disclosed herein can also be blocked with an aminoalkyl group, e.g., a 5′-O-alkylamino substituent. Other 5′ conjugates can inhibit 5′-3′ exonucleolytic cleavage. A 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

The miRNA inhibitors disclosed herein can also be attached to a peptide or a peptidomimetic ligand which may affect pharmacokinetic distribution of the miRNA inhibitor such as by enhancing cellular recognition, absorption and/or cell permeation. The peptide or peptidomimetic moiety can be about 5-50 amino acids long, e.g., about 5, 10, 15, 20, 25, 30, 35, 40, 45, or 50 amino acids long. A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724 (2003)).

### MiR-155 Expression Vectors

Expression vectors that contain a miR-155 or anti-miR-155 coding sequence are used in some embodiments for delivery of a miR-155 or anti-miR155 to target cells. Expression vectors that contain a miR-155 sequence and/or anti-miR-155 sequence may optionally be associated with one or more regulatory elements that direct the expression of the coding sequence in a target cell. MiR-155 and anti-miR-155 sequences are described in detail in the previous section. The choice of vector and/or expression control sequences to which the encoding sequence is operably linked depends directly, as is well known in the art, on the functional properties desired, e.g., miRNA transcription, and the host cell to be transformed.

In some embodiments an expression vector is capable of directing replication in an appropriate host and of expression of a miR-155 or anti-miR-155 in a target cell. Vectors that can be used are well known in the art and include, but are not limited to, pUC8, pUC9, pBR322 and pBR329 available from BioRad Laboratories, (Richmond, Calif.), pPL and pKK223 available from Pharmacia (Piscataway, N.J.) for use in prokaryotic cells, and pSVL and pKSV-10 (Pharmacia), pBPV-1/pML2d (International Biotechnologies, Inc.), pcDNA and pTDT1 (ATCC, #31255), for use in eukaryotic cells, as well as eukaryotic viral vectors such as adenoviral or retroviral vectors.

Vectors may include a selection gene whose expression confers a detectable marker such as a drug resistance. Typical selection genes encode proteins that confer resistance to antibiotics or other toxins, e.g., ampicillin, neomycin, methotrexate, or tetracycline, complement auxotrophic deficiencies, or supply critical nutrients withheld from the media. Such selection systems are well known in the art. The selectable marker can optionally be present on a separate plasmid and introduced by co-transfection.

Expression control elements that are used for regulating the expression of an operably linked coding sequence are known in the art and include, but are not limited to, inducible promoters, constitutive promoters, enhancers, and other regulatory elements. In some embodiments an inducible promoter is used that is readily controlled, such as being responsive to a nutrient in the target cell's medium. In some embodiments, the promoter is the U6 promoter or CMV promoter.

Other methods, vectors, and target cells suitable for adaptation to the expression of miR-155 in target cells are well known in the art and are readily adapted to the specific circumstances.

### Delivery of Oligonucleotides and Expression Vectors to a Target Cell or Tissue

In some embodiments, a miR-155 or anti-miR-155 oligonucleotide is delivered to a target cell, tissue or organ. In other embodiments, an expression vector encoding a miR-155 or anti-miR-155 is delivered to a target cell, tissue or organ where the miR-155 or anti-miR-155 is expressed. In some embodiments, delivery is systemic and the oligonucleotide or expression vector is taken up into target cells where it has a desired activity. In some such embodiments, the oligonucleotide or expression vector may be taken up by non-target cells or tissues, but preferably does not have a significant negative effect on such cells or tissues, or on the organism as a whole.

Methods for delivery of oligonucleotides and expression vectors to target cells are well known in the art and exemplary methods are described briefly below. Target cells can be, for example, immune cells, such as immune cells involved in autoimmune disorders. In some embodiments target cells are T cells, such as CD4+ T cells. In some embodiments the CD4+ T cells are T_{H}17 and/or T_{H}1 T cells.

In some embodiments target immune cells are dendritic cells.

Target cells may be present in a host, such as in a mammal, or may be in culture outside of a host. Delivery of miR-155 or anti-miR-155 to target cells in vivo, ex vivo and in vitro is contemplated, depending on the particular circumstances.

In some embodiments, a miR-155 or anti-miR-155 oligonucleotide, or expression vector encoding such an oligonucleotide, is delivered to a target organ or tissue. Target organs and tissues may include locations where immune cells, such as CD4+ cells or dendritic cells, or precursors of such cells, are known to be located. Examples include, without limitation, the peritoneal cavity, spleen, lymph nodes, including mesenteric lymph nodes and peripheral lymph nodes, thymus, and bone marrow. In some embodiments the target tissue is a tissue (or organ) undergoing inflammation. Tissue that may be undergoing inflammation is not limited in any way and may be, for example, tissues of the central nervous system, skin, intestines, joints, kidneys and the like. In some embodiments the target tissue is a tissue undergoing autoimmune inflammation. In some embodiments, miR-155 or anti miR-155 oligonucleotides are delivered to the spleen or lymph nodes, or to precursor cells, such as hematopoietic stem cells in the bone marrow or elsewhere. In some embodiments, miR-155 or anti miR-155 oligonucleotides are delivered to tissues undergoing autoimmune inflammation. In some embodiments miR-155 or anti-miR-155 are delivered systemically, such as by intravenous injection. Additional routes of administration may include, for example, oral, topical, intrathecal, intraperitoneal, intranasal, intraocular, and intramuscular. Other routes of administration are well known in the art and will be apparent to the skilled artisan.

Delivery of oligonucleotides and/or expression vectors to a target cell or tissue can be achieved in a variety of ways. In some embodiments, a transfection agent is used. A transfection agent, or transfection reagent or delivery vehicle, is a compound or compounds that bind(s) to or complex(es) with oligonucleotides and polynucleotides, and enhances their entry into cells. Examples of transfection reagents include, but are not limited to, cationic liposomes and lipids, polyamines, calcium phosphate precipitates, polycations, histone proteins, polyethylenimine, polylysine, and polyampholyte complexes. Transfection reagents are well known in the art. One transfection reagent suitable for delivery of miRNA is siPORT™ NeoFX™ transfection agent (Ambion), which can be used to transfect a variety of cell types with miRNA. miRNAs can be readily electroporated into primary cells without inducing significant cell death. In addition, miRNAs can be transfected at different concentrations. The transfection efficiency of synthetic miRNAs has been shown to be very good, and around 100% for certain cell types (Ambion® miRNA Research Guide, page 12. See also, www.ambion.com/miRNA).

Reagents for delivery of miRNA, anti-miRNA and expression vectors can include, but are not limited to protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associate functional groups with a polynucleotide. Functional groups can include cell targeting moieties, cell receptor ligands, nuclear localization signals, compounds that enhance release of contents from endosomes or other intracellular vesicles (such as membrane active compounds), and other compounds that alter the behavior or interactions of the compound or complex to which they are attached (interaction modifiers). For delivery in vivo, complexes made with sub-neutralizing amounts of cationic transfection agent may be preferred.

In some embodiments, polycations are mixed with polynucleotides for delivery to a cell. Polycations are a very convenient linker for attaching specific receptors to DNA and as result, DNA/polycation complexes can be targeted to specific cell types. Here, targeting is preferably to cells involved in innate immunity. An endocytic step in the intracellular uptake of DNA/polycation complexes is suggested by results in which functional DNA delivery is increased by incorporating endosome disruptive capability into the polycation transfection vehicle. Polycations also cause DNA condensation. The volume which one DNA molecule occupies in complex with polycations is drastically lower than the volume of a free DNA molecule. The size of DNA/polymer complex may be important for gene delivery in vivo. In some embodiments, miR-155 or anti-miR-155 nucleic acids and a transfection reagent are delivered systematically such as by injection. In other embodiments, they may be injected into particular areas comprising target cells, such as particular organs, for example the bone marrow.

Polymer reagents for delivery of miRNA, anti-miRNA and expression vectors may incorporate compounds that increase their utility. These groups can be incorporated into monomers prior to polymer formation or attached to polymers after their formation. A miRNA, anti-miRNA or expression vector transfer enhancing moiety is typically a molecule that modifies a nucleic acid complex and can direct it to a cell location (such as tissue cells) or location in a cell (such as the nucleus) either in culture or in a whole organism. By modifying the cellular or tissue location of the complex, the desired localization and activity of the miRNA, anti-miRNA or expression vector can be enhanced. The transfer enhancing moiety can be, for example, a protein, peptide, lipid, steroid, sugar, carbohydrate, nucleic acid, cell receptor ligand, or synthetic compound. The transfer enhancing moieties can enhance cellular binding to receptors, cytoplasmic transport to the nucleus and nuclear entry or release from endosomes or other intracellular vesicles.

Nuclear localizing signals can also be used to enhance the targeting of the miRNA, anti-miRNA or expression vector into proximity of the nucleus and/or its entry into the nucleus. Such nuclear transport signals can be a protein or a peptide such as the SV40 large Tag NLS or the nucleoplasmin NLS. These nuclear localizing signals interact with a variety of nuclear transport factors such as the NLS receptor (karyopherin alpha) which then interacts with karyopherin beta. The nuclear transport proteins themselves could also function as NLS's since they are targeted to the nuclear pore and nucleus.

Compounds that enhance release from intracellular compartments can cause DNA release from intracellular compartments such as endosomes (early and late), lysosomes, phagosomes, vesicle, endoplasmic reticulum, Golgi apparatus, trans Golgi network (TGN), and sarcoplasmic reticulum and could be used to aid delivery of miRNA-155 or anti-miR-155. Release includes movement out of an intracellular compartment into cytoplasm or into an organelle such as the nucleus. Such compounds include chemicals such as chloroquine, bafilomycin or Brefeldin Al and the ER-retaining signal (KDEL sequence), viral components such as influenza virus hemagglutinin subunit HA-2 peptides and other types of amphipathic peptides.

Cellular receptor moieties are any signal that enhances the association of the miRNA, anti-miRNA or expression vector with a cell. Enhanced cellular association can be accomplished by either increasing the binding of the polynucleotide or polynucleotide complex to the cell surface and/or its association with an intracellular compartment, for example: ligands that enhance endocytosis by enhancing binding the cell surface. Cellular receptor moieties include agents that target to asialoglycoprotein receptors by using asialoglycoproteins or galactose residues. Other proteins such as insulin, EGF, or transferrin can be used for targeting. Peptides that include the RGD sequence can also be used to target many cells. Chemical groups that react with sulfhydryl or disulfide groups on cells can also be used to target many types of cells. Folate and other vitamins can also be used for targeting. Other targeting groups include molecules that interact with membranes such as lipids fatty acids, cholesterol, dansyl compounds, and amphotericin derivatives. In addition viral proteins could be used to target cells.

The skilled artisan will be able to select and use an appropriate system for delivering miRNA-155, anti-miRNA-155 or an expression vector to target cells in vitro or in vivo without undue experimentation.

### Modulation of Inflammatory T Cell Production and Development

miRNA-155 can be used to modulate production and/or development of inflammatory immune cells, particularly CD4+ T cells, and, in some embodiments T_{H}17 T cells and/or T_{H}1 T cells. Development and activity of CD4+ T cells can be modulated by administering a miR-155 oligonucleotide or anti-miR-155, such as an antisense miR-155 oligonucleotide, to these cells or precursor cells, such as hematopoietic stem cells, or to a tissue, organ or organism comprising these cells. In some embodiments a miR-155 oligonucleotide or anti-miR-155, such as an antisense miR-155 oligonucleotide, is administered to hematopoietic stem cells, lymphoid progenitor cells and/or developed T_{H}1 and T_{H}17 cells. The cells may be in vivo, such as in a mammal, ex vivo or in vitro.

The absence of miR-155 or miR-155 activity can block or reduce development of T_{H}1 and/or T_{H}17 inflammatory T cells, for example in the context of autoimmunity. In some embodiments, anti-miR-155, such as an antisense miR-155 oligonucleotide, is administered to block or reduce development of T_{H}1 and/or T_{H}17 inflammatory T cells. For example, the anti-miR-155 may be administered to a tissue undergoing inflammation due to autoimmunity in order to reduce or block development of T_{H}1 and/or T_{H}17 T cells.

In some embodiments, modulation of CD4+ T cell production may comprise, for example, an increase or decrease in CD4+ T cell proliferation, or an increase or decrease in total CD4+ T cell number, such as an increase or decrease of T_{H}17 and/or T_{H}1 T cells, or a change in the percentage of CD4+ T cells in a population. In some embodiments, modulation in the development of CD4+ T cells may be reflected in an increase or decrease in the CD4+ T cell driven recall response to an antigen. Exemplary ways of measuring modulation of inflammatory T cell production and development are provided in the Examples below.

In some embodiments, production, development, and/or activity of CD4+ T cells, such as T_{H}17 and/or T_{H}1 T cells, can be increased by administering miRNA-155 to a CD4+ T cell population. In some embodiments the miR-155 is delivered directly to CD4+ T cells. In some embodiments the miR-155 is delivered to organs or tissues comprising CD4+ T cells, such as the lymph nodes or spleen. In some embodiments the miR-155 is delivered to a precursor cell, such as hematopoietic stem cell, or to a tissue comprising precursor cells, such as bone marrow. In some embodiments the miR-155 is delivered to a mammal comprising CD4+ T cells. In some embodiments the CD4+ T cells are T_{H}17 and/or T_{H}1 T cells.

In some embodiments, a miR-155 oligonucleotide is administered using an expression vector, as described herein. In some embodiments, the miR-155 expression vector comprises a nucleic acid sequence encoding a miRNA-155 operably linked to a U6 promoter or a CMV promoter.

Increased numbers of CD4+ T cells can be detected, for example, by FACS analysis after administering a miRNA-155 oligonucleotide or a miRNA-155 expression vector to CD4+ T cells and/or a target tissue, organ, or organism.

In other embodiments, production, development, and/or activity of CD4+ T cells is downregulated by administering an anti-miR-155, such as an antisense miRNA-155 oligonucleotide, to a CD4+ T cell population. In some embodiments the CD4+ T cells are T_{H}17 T cells and/or T_{H}1 T cells.

In some embodiments the anti-miR-155 is delivered directly to CD4+ T cells. In some embodiments the anti-miR-155 is delivered to organs or tissues comprising CD4+ T cells, such as the lymph nodes or spleen. In some embodiments the anti-miR-155 is delivered to precursor cells, such as hematopoietic stem cells, or to a tissue comprising precursor cells, such as bone marrow. In some embodiments the anti-miR-155 may be delivered to an organism. In some embodiments the CD4+ T cells are T_{H}17 and/or T_{H}1 T cells.

In some embodiments, an antisense miR-155 oligonucleotide is administered to CD4+ T cells using an expression vector, as described herein. In some embodiments, the antisense miR-155 expression vector comprises a nucleic acid sequence encoding an antisense miRNA-155 operably linked to a U6 promoter or a CMV promoter.

Decreased numbers of CD4+ T cells can be detected, for example, by FACS analysis after administering an antisense miRNA-155 oligonucleotide or an antisense miRNA-155 expression vector to a target tissue.

In some embodiments, development of T_{H}17 and/or T_{H}1 CD4+ T cells is downregulated by administering an antisense miRNA-155 oligonucleotide to CD4+ T cells or precursors of CD4+ T cells. In other embodiments, the methods comprise administering an antisense miRNA-155 expression vector and expressing a miRNA-155 in the target.

In some embodiments an excess of miR-155 antisense oligonucleotide is provided, relative to the amount of miR-155 expected to be expressed endogenously.

Any of a variety of miRNA-155 or antisense miRNA-155 molecules can be used to regulate development, production and/or activity of CD4+ T cells in the embodiments described above. In some embodiments, a miR-155 oligonucleotide comprises all or a portion of miR-155, pre-miR-155, or a miR-155 seed sequence, or a variant thereof. Mixtures of various miR-155 nucleic acids can also be used.

In some embodiments, a miR-155 expression vector comprises a sequence encoding a miRNA-155 or a variant thereof.

As described above, in some embodiments, an antisense miR-155 oligonucleotide is used. In some embodiments, an antisense miR-155 expression vector comprises a sequence encoding an antisense miRNA-155 oligonucleotide.

In some embodiments, to regulate development, production and/or activity of certain immune cells, such as CD4+ T cells, miRNA-155 or antisense miRNA-155 are delivered to hematopoietic tissues. For example, in some embodiments miR-155 oligonucleotides or expression vectors, or anti-miR-155 oligonucleotides or expression vectors, are delivered to the bone marrow. In some embodiments, delivery is to tissues or organs comprising immune cells. In other embodiments the miR-155 or anti-miR-155 is delivered directly to the immune cells to be regulated or to precursor cells, such as hematopoietic stem cells.

miRNA-155 or antisense miRNA-155 can be delivered as described herein or as known in the art. For example, delivery can be achieved by modification of an oligonucleotide encoding a miR-155 with cholesterol to help it easily penetrate the cell membrane. Delivery can be optimized by using modified nucleotides or utilizing backbone modifications. Delivery can be achieved by injection into particular areas such as hematopoietic tissue or the bone marrow.

In other embodiments, miR-155, anti-miR-155 or expression vectors are delivered systemically. miRNA-155 or antisense miRNA-155 can be delivered in combination with pharmaceutically acceptable carriers. In some embodiments miRNA-155 or antisense miRNA-155 or expression vectors encoding miR-155 or antisense miR-155 can be injected intravenously.

In some embodiments, proliferation of inflammatory immune cells, particularly CD4+ T cells, can be used to measure the effect of miR-155 or antisense miR-155 on CD4+ T cells. For example, proliferation of T_{H}17 and/or T_{H}1 CD4+ cells can be measured. Measurements of proliferation can take place in an appropriate spot, such as in the bone marrow, thymus, spleen, periphery, peritoneal cavity or lymph nodes, such as peripheral lymph nodes or mesenteric lymph nodes. Measurement of proliferation can be by any method known in the art, for example by FACS analysis.

### Modulation of Cytokine Production

miRNA-155 can be used to regulate production of cytokines by dendritic cells (DCs). In some embodiments the cytokines promote T_{H}17 cell formation. In some embodiments production of cytokines by DCs is regulated using miR-155 or anti-miR-155. Cytokines that can be regulated include, for example, IL23/IL-17, G-CSF, IL-6, IFNγ and TNF-α.

In some embodiments production of one, two, three, four or all five of IL23/IL-17, G-CSF, IL-6, IFNγ and TNF-α by DCs is downregulated by inhibiting miRNA-155 activity in the DCs, for example by delivering antisense miR-155 to the DCs. In other embodiments, production of one, two, three, four or all five of these cytokines by DCs is increased by delivering miR-155 to the DCs.

In some embodiments, production of one or more cytokines by dendritic cells is decreased by decreasing miR-155 activity, for example by delivering antisense miRNA-155 oligonucleotides to DCs in which cytokine production is to be reduced. The cytokines may be cytokines that promote T_{H}17 cell formation. In some embodiments the cytokines may be selected from IL23/IL-17, G-CSF, IL-6, IFNγ and TNF-α. In some embodiments, the antisense miR-155 oligonucleotide is complementary to all or a portion of mature miR-155, pre-miR-155, pri-miR-155, or a miR-155 seed sequence. Mixtures of various antisense miR-155 nucleic acids can also be used.

In some embodiments, antisense miR-155 is delivered by methods comprising administering an antisense miRNA-155 expression vector to DCs and expressing the antisense miRNA-155 in the dendritic cells, thereby reducing production of one or cytokines selected from IL23/IL-17, G-CSF, IL-6, IFNγ and TNF-α. In some embodiments, the antisense miR-155 expression vector comprises a nucleic acid sequence encoding an antisense miRNA-155 operably linked to a U6 promoter or a CMV promoter.

To reduce production of certain cytokines, antisense miRNA-155 or expression vectors encoding the antisense miR-155 can be delivered to directly to dendritic cells or to tissue comprising dendritic cells. In other embodiments the antisense miR-155 or expression vector is delivered to precursor cells that develop into dendritic cells. In some embodiments antisense miR-155 or anti-miR-155 expression vector is delivered to tissues comprising precursor cells, such as bone marrow. In some embodiments antisense miR-155 or anti-miR-155 expression vector is delivered systemically.

In other embodiments, production of cytokines by immune cells can be increased by enhancing miR-155 activity, such as by delivering miR-155 or a miR-155 expression vector to the DCs. The miR-155 can be essentially as described elsewhere herein.

In some embodiments production of one, two, three, four or all five of IL23/IL-17, G-CSF, IL-6, IFNγ and TNF-α by DCs is increased by enhancing miRNA-155 activity in the DCs, for example by delivering miR-155 to the DCs.

In some embodiments, the methods comprise administering a miRNA-155 oligonucleotide to DCs in which cytokine production is to be increased. In some embodiments, the miR-155 oligonucleotide comprises all or a portion of mature miR-155, pre-miR-155, pri-miR-155, or a miR-155 seed sequence, or a variant thereof. Mixtures of various miR-155 nucleic acids can also be used. In some embodiments, the miR-155 comprises a sequence selected from the group consisting of SEQ ID NOs: 1-5, or a variant thereof.

In other embodiments, the methods comprise administering a miRNA-155 expression vector to DCs and expressing the miRNA-155 in the dendritic cells to increase production of one or more cytokines selected from IL23/IL-17, G-CSF, IL-6, IFNγ and TNF-α. In some embodiments, the miR-155 expression vector comprises a nucleic acid sequence encoding a miRNA-155 nucleic acid operably linked to a U6 promoter or a CMV promoter.

To enhance production of certain cytokines, such as one or more of IL23/IL-17, G-CSF, IL-6, IFNγ and TNF-α, miRNA-155 or an expression vector encoding miR-155 can be delivered directly to dendritic cells. In some embodiments, miR-155 or the expression vector is delivered to tissue comprising dendritic cells. In other embodiments the miR-155 or the expression vector is delivered to precursor cells that develop into dendritic cells or tissues comprising precursor cells, such as bone marrow. In some embodiments miR-155 or the expression vector is delivered to tissues comprising the dendritic cells. In some embodiments miR-155 or the expression vector is delivered systemically.

In general, miRNA-155 or antisense miRNA-155 can be delivered as described herein or as known in the art. For example, in some embodiments an miR-155 or antisense miR-155 oligonucleotide or expression vector can be administered to the cells by transfection. In other embodiments, they may be directly injected into bone marrow. miRNA-155 or antisense miRNA-155 can be modified to enhance delivery. For example, these oligonucleotides can be modified with cholesterol. In other embodiments miRNA-155 or antisense miRNA-155 can be injected into target cells, a tissue comprising the target cells, or injected systemically into a mammal comprising the target cells. miRNA-155 or antisense miR-155 can also be coupled to a ligand of a target cell surface receptor and enter into target cells through endocytosis.

### Methods of Reducing Inflammation

In some embodiments, methods of reducing T cell dependent tissue inflammation are provided. T cell dependent tissue inflammation is down-regulated by inhibiting miRNA-155 activity in the T cells, for example by delivering an anti-miR-155, such as an antisense miR-155 oligonucleotide, to the T cells. In some embodiments, T cell dependent tissue inflammation is decreased by delivering antisense miRNA-155 oligonucleotides to a population of T cells, thereby decreasing miR-155 activity. The T cells may be, for example, T_{H}1 and/or T_{H}17 T cells. In some embodiments, the anti-miR-155 is delivered to an inflamed tissue, such as a tissue undergoing autoimmune inflammation. In some embodiments the anti-miR-155 is delivered to T cells related to the autoimmune inflammation, such as T_{H}1 and/or T_{H}17 T cells. Mixtures of various antisense miR-155 nucleic acids can also be used. In some embodiments, the miR-155 antisense comprises a sequence that is complementary to, or is able to hybridize to (for example under stringent conditions), all or a portion of a nucleic acid selected from the group consisting of SEQ ID NOs: 1-5.

In other embodiments, antisense miR-155 is delivered by methods comprising administering an antisense miRNA-155 expression vector to the T cells and expressing the antisense miRNA-155 in the T cells, thereby reducing T cell mediated inflammation. In some embodiments, the antisense miR-155 expression vector comprises a nucleic acid sequence encoding an antisense miRNA-155 nucleic acid operably linked to a U6 promoter or a CMV promoter.

In some embodiments the antisense miR-155 or antisense miR-155 expression vector is delivered directly to CD4+ T cells, such as T_{H}1 and/or T_{H}17 T cells. In some embodiments the antisense miR-155 or the expression vector is delivered to organs or tissues comprising CD4+ T cells, such as the lymph nodes or spleen. In some embodiments the antisense miR-155 or expression vector is delivered to the inflamed tissue. In some embodiments the anti-miR-155 or expression vector is delivered to a precursor cell, such as hematopoietic stem cell, or to a tissue comprising precursor cells, such as bone marrow. In some embodiments the CD4+ T cells are T_{H}17 and/or T_{H}1 T cells. In some embodiments the anti-miR-155 is delivered directly to CD4+ T cells. In some embodiments the anti-miR-155 is delivered to organs or tissues comprising CD4+ T cells, such as the lymph nodes or spleen. In some embodiments the anti-miR-155 is delivered to a precursor cell, such as hematopoietic stem cell, or to a tissue comprising precursor cells, such as bone marrow. In some embodiments the CD4+ T cells are T_{H}17 and/or T_{H}1 T cells.

In some embodiments the T cell mediated tissue inflammation is tissue specific autoimmune inflammation. In some embodiments the T cell mediated tissue inflammation is chronic inflammation directed at tissue specific antigens.

After delivery of anti-miR-155, a decrease in inflammation, such as autoimmune inflammation, may be measured. In some embodiments, a reduction in proliferation of CD4+ T cells may be measured.

### Methods of Increasing Immune Response to Infection

In some embodiments, methods of increasing the immune response to infection in a mammal are provided. For example, the immune response to pathogens, such as viruses and bacteria, can be increased. In some embodiments, a subject, preferably a mammal, suffering from infection by a pathogen or at risk of infection by a pathogen is identified. A miR-155 nucleic acid is administered to the subject. The miR-155 may be delivered alone, in a modified form, or as a miR-155 expression vector. In some embodiments, a miR-155 expression vector comprises a nucleic acid sequence encoding a miRNA-155, such that the miR-155 is expressed in the desired cells.

In some embodiments the miR-155 is delivered directly to CD4+ T cells. In some embodiments the miR-155 is delivered to organs or tissues comprising CD4+ T cells, such as the lymph nodes or spleen. In some embodiments the miR-155 is delivered to a precursor cell, such as hematopoietic stem cell, or to a tissue comprising precursor cells, such as bone marrow. In some embodiments the CD4+ T cells are T_{H}17 and/or T_{H}1 T cells. In other embodiments, the miR-155 is delivered systemically, such as by intravenous injection.

In some embodiments a patient is identified that is suffering from or at risk of suffering from infection by a pathogen. A miR-155 oligonucleotide or expression vector encoding a miR-155 oligonucleotide is administered to the patient, thereby increasing the immune response to the pathogen. In some embodiments, the miR-155 oligonucleotide or expression vector is delivered to a tissue associated with the production of immune cells, such as the spleen or lymph nodes. In other embodiments delivery may be to bone marrow. In other embodiments, delivery may be systemic, such as by intravenous injection. An increase in immune response may be measured by methods disclosed herein, or known in the art. For example, expression of inflammatory cytokines may be measured. In some embodiments, proliferation of CD4+ T cells is measured in the patient. A reduction in symptoms associated with the infection may also be measured.

### Methods of Treatment

In some embodiments, methods of treating autoimmune inflammation are provided. For example, autoimmune diseases such as rheumatoid arthritis, lupus, multiple sclerosis, inflammatory bowel disease and psoriasis may be treated. In some embodiments an autoimmune disorder involving T cell dependent inflammation, such as T_{H}1 and/or T_{H}17 cell dependent inflammation, is treated.

A subject is identified that is suffering from an autoimmune disease, or that would otherwise benefit from enhanced CD4+ T cell development, proliferation and/or activation, is identified. An anti-miRNA-155, such as an antisense miRNA oligonucleotide is administered to the subject. The antisense miR-155 oligonucleotide may be delivered alone, in a modified form, or as an antisense miR-155 expression vector. In some embodiments, an antisense miR-155 expression vector comprises a nucleic acid sequence encoding an antisense miRNA-155 oligonucleotide, such that the antisense miR-155 oligonucleotide is expressed in T cells in the patient.

In some embodiments, administration of an anti-miR-155 reduces inflammatory cytokines, such as IL-23/IL-17, GM-CSF, IL-6, IFNγ and TNF-α. Production of these and other inflammatory cytokines may be measured, before, after or before and after administration of the anti-miR-155. In addition, symptoms of the autoimmune disease, or other markers of the disease may be measured before and/or after administration of the anti-miR-155, for example to determine its efficacy. In some embodiments, a reduction in proliferation of CD4+ T cells may be measured. In some embodiments, development of T_{H}1 and/or T_{H}17 cells is reduced.

In some embodiments the antisense miR-155 oligonucleotide is delivered directly to a CD4+ T cell population. In some embodiments the antisense miR-155 oligonucleotide is delivered to organs or tissues comprising CD4+ T cells, such as the lymph nodes or spleen. In some embodiments the antisense miR-155 oligonucleotide is delivered to a precursor cell, such as a hematopoietic stem cell, or to a tissue comprising precursor cells, such as bone marrow. In other embodiments, an antisense miR-155 oligonucleotide is delivered systemically, such as by intravenous injection. An appropriate dose of a miR-155 oligonucleotide or other anti-miR-155 can be determined by the skilled artisan in view of the particular circumstances.

The following examples are offered by way of illustration and not by way of limitation.

### EXAMPLES

The role of miR-155 during autoimmune inflammatory disease was investigated. As discussed in the Examples below, mice that are lacking miR-155 (miR-155−/−) are highly resistant to experimental autoimmune encephalomyelitis (EAE) due to a lack of miR-155 function in the hematopoietic compartment. These mice have a deficit in their production of inflammatory T cells including the T_{H}17 and T_{H}1 subsets that mediate disease. Consistent with this, miR-155 acts in a T cell-intrinsic manner to drive development of inflammatory T cells in vivo, and can enhance dendritic cell (DC) production of cytokines that promote T_{H}17 cell formation. Thus, one aspect of miR-155 function is the promotion of T cell-dependent tissue inflammation. In view of this activity miR-155 can be used as a target for the treatment of autoimmune disorders.

### Example 1

### miR-155−/− Mice are Resistant to EAE Induced by MOG₃₅₋₅₅

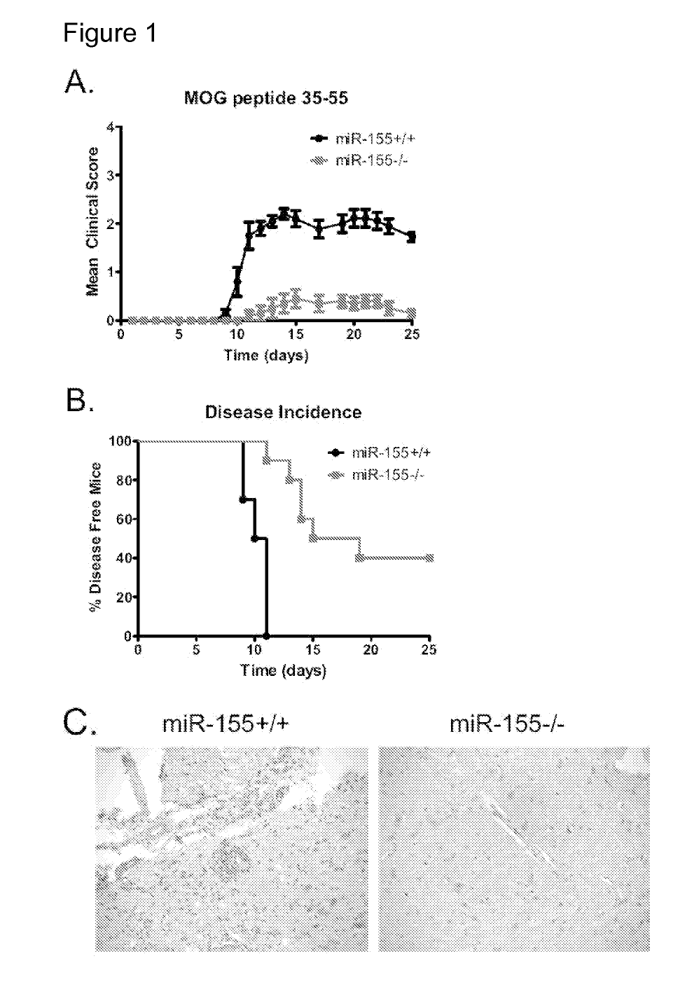

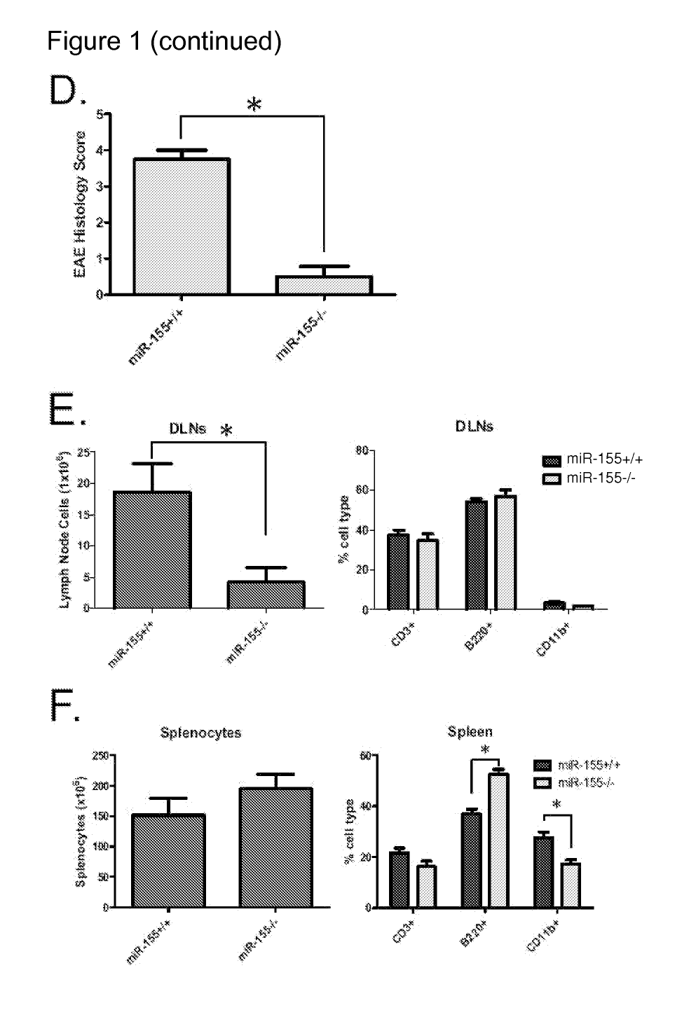

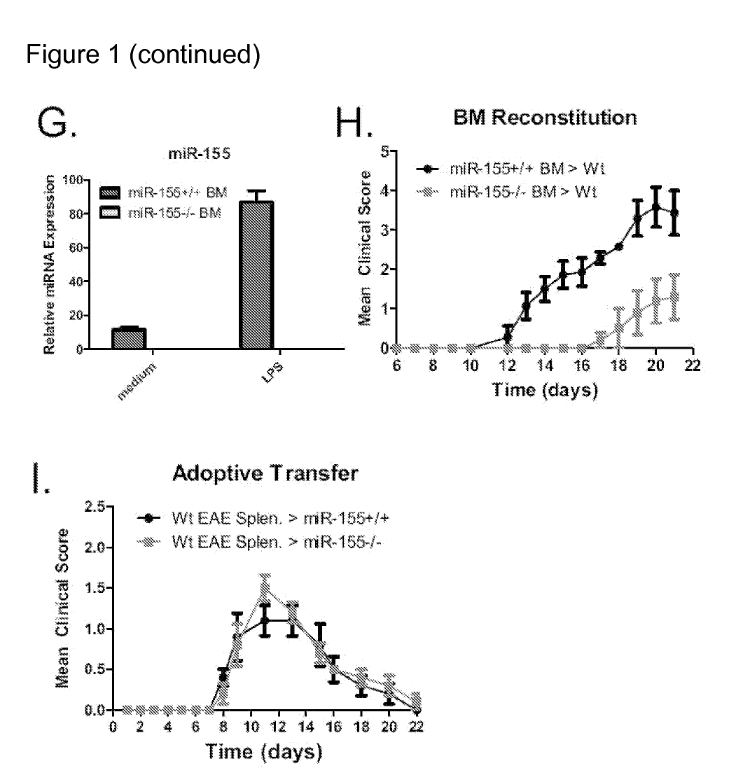

To identify a possible role for miR-155 in mediating tissue specific autoimmune inflammation, a mouse model of EAE was used. Both Wt and miR-155−/− mice were immunized with 100 μg of the myelin oligodendrocyte glycoprotein (MOG) peptide₃₅₋₅₅ emulsified in complete Freund's adjuvant (CFA) followed by administration of pertussis toxin. As anticipated, Wt mice first displayed neurologic symptoms approximately 9 days post-immunization, with peak disease severity on day 14 (average clinical score of 2.1), and 100% disease incidence (FIGS. 1A and 1B). In contrast, miR-155 deficient mice exhibited a later onset of symptoms on day 11, with a low peak disease severity on day 15 (average clinical score of 0.3). Unlike the Wt controls, the disease incidence in miR-155−/− mice was only 60% (FIGS. 1A and 1B). On day 25, mice were sacrificed and underwent further evaluation including tissue histological analysis. H&E brain cross-sections were scored for disease severity by a pathologist blinded to the genotype of the tissue under analysis (FIGS. 1C and 1D). As expected, Wt mice suffered from heavy perivascular congestion, parenchymal infiltration and focal meningeal lymphocytosis. However, brain tissue from miR-155−/− mice showed minimal histologic evidence of inflammation consistent with the mild clinical manifestation of EAE (FIGS. 1C and 1D). Furthermore, analysis of the draining lymph nodes (DLNs) and spleens showed decreased overall cellularity in DLNs in miR-155−/− mice, and compositionally fewer CD11b+ myeloid cells in miR-155−/− spleens (FIGS. 1E and 1F). These observations indicate a reduced inflammatory condition in miR-155−/− mice.

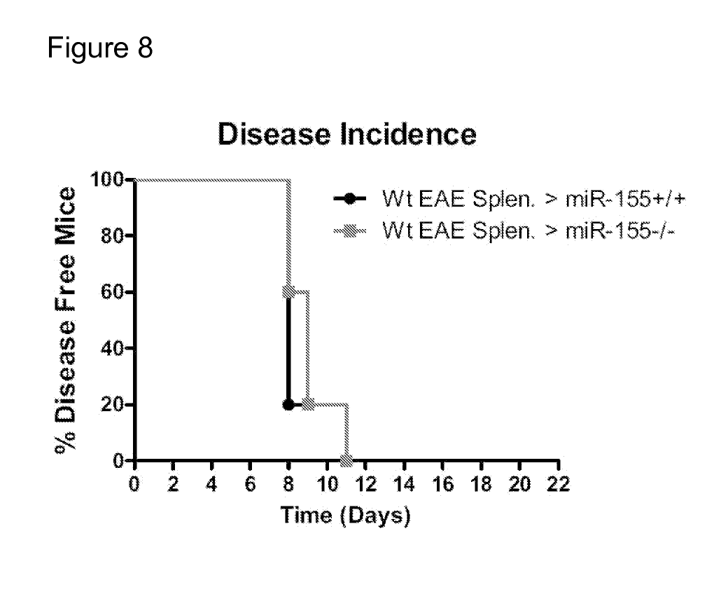

Lethally irradiated Wt C57BL6 mice were reconstituted with either miR-155+/+ or miR-155−/− BM cells. After 4 months, proper engraftment and localization of the miR-155 deficiency to the hematopoietic compartment was confirmed by assaying miR-155 in activated splenic B cells (FIG. 1G). Following induction of EAE, mice with miR-155+/+ hematopoietic cells exhibited a much faster and more severe disease phenotype than mice containing miR-155−/− hematopoietic cells (FIG. 1H). In a separate experiment, 25×10⁶ Wt encephalitogenic splenocytes from day 12 EAE Wt mice were transferred into Wt or miR-155−/− hosts, which were monitored for the presence of clinical symptoms. Both groups began to show symptoms by day 8 post-adoptive transfer, and had comparable disease scores throughout the 22 day time course (FIG. 1I). Furthermore, both cohorts had a disease incidence of 100% (FIG. 8). Taken together, these data demonstrate that miR-155 functions in the hematopoietic compartment to promote inflammation, such as EAE.

### Example 2

### miR-155−/− Mice Exhibit Defective Inflammatory T Cell Development During EAE

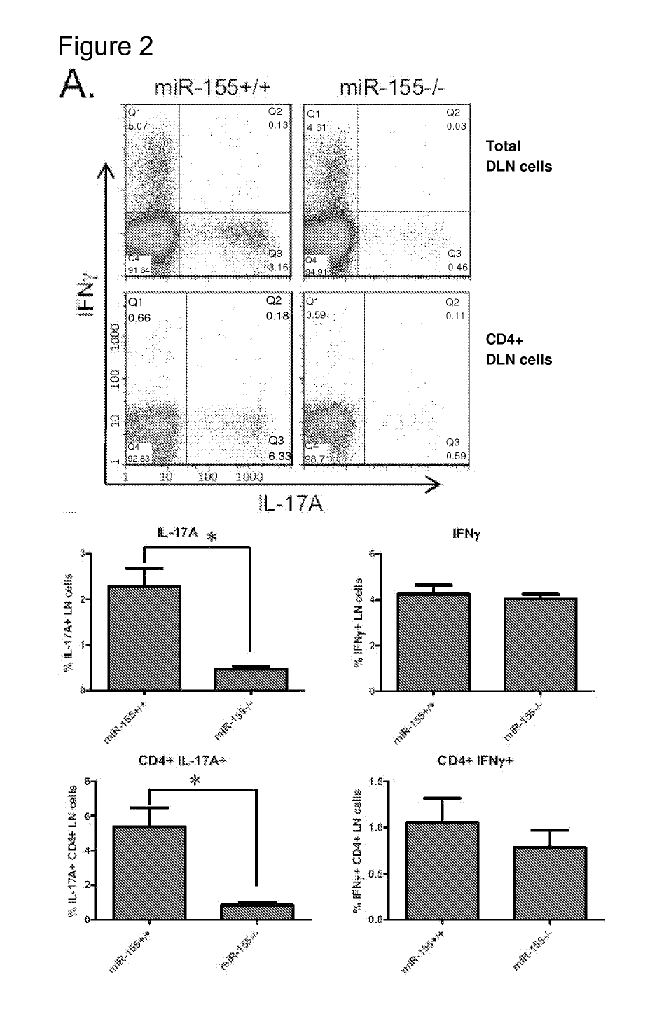

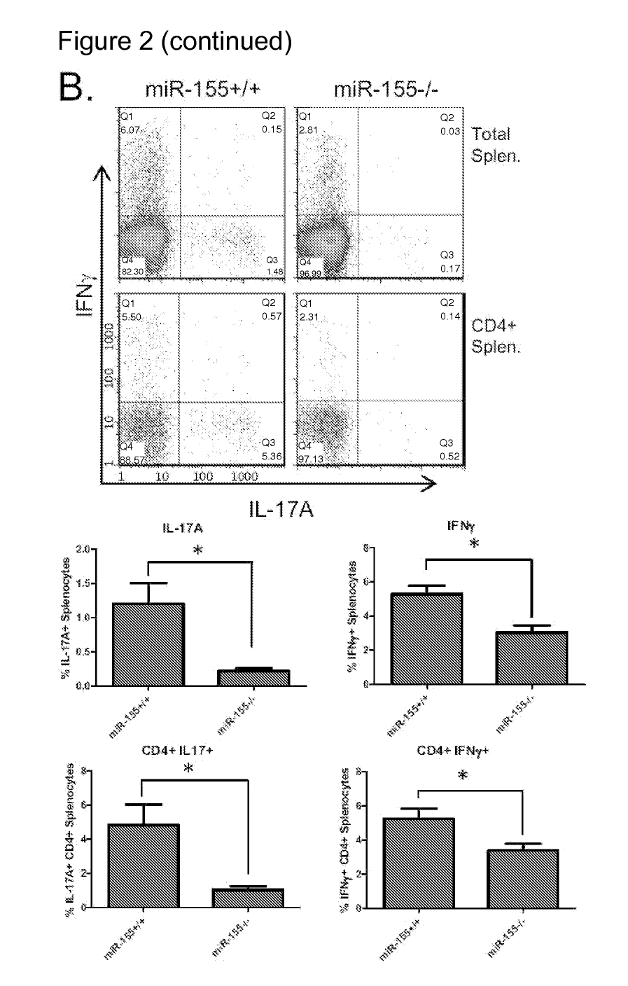

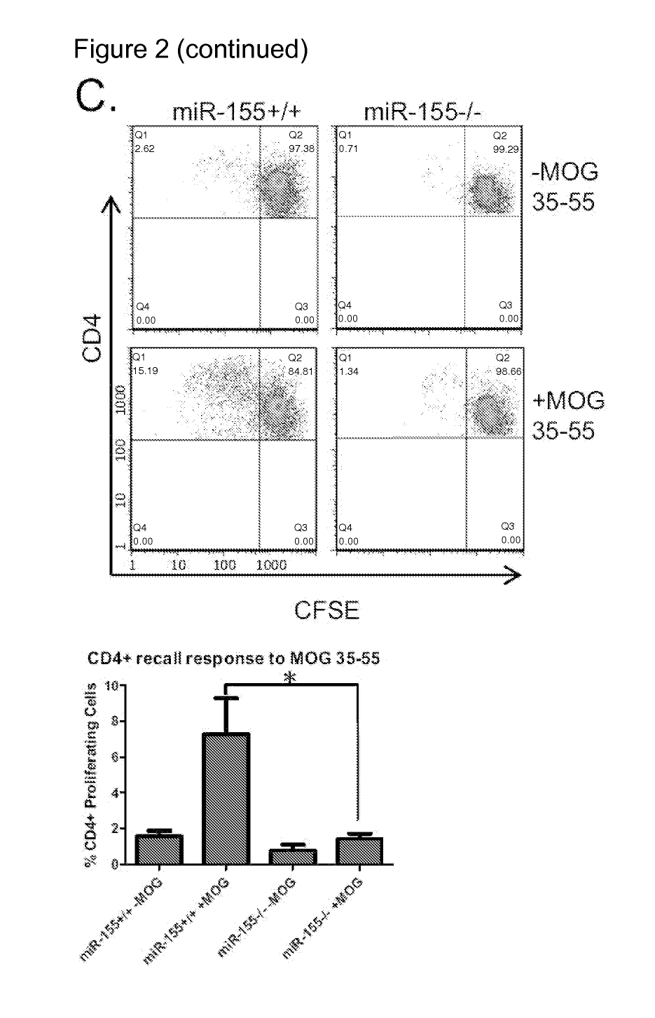

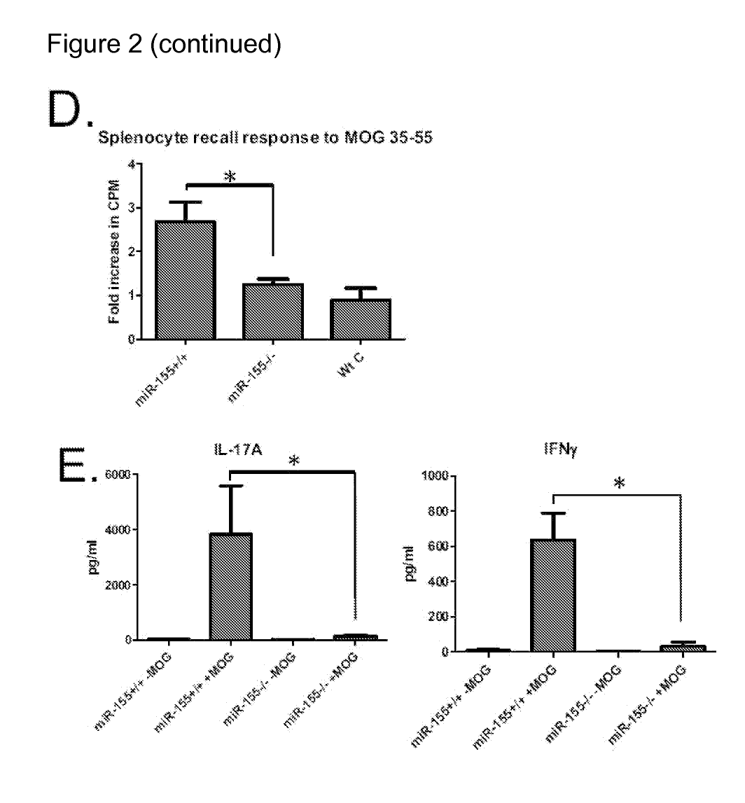

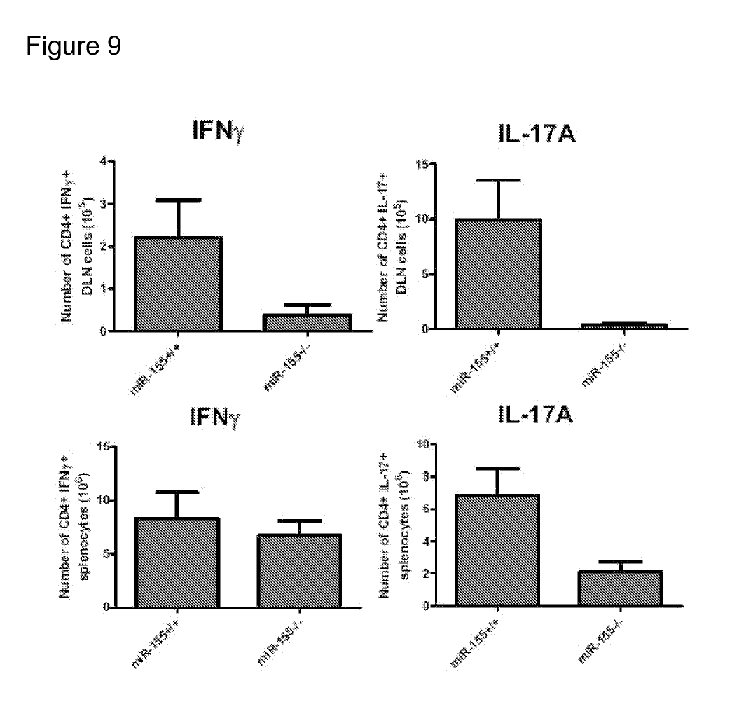

T_{H}17 and T_{H}1 cells are hematopoietic cells that develop during tissue specific inflammatory responses and play a pivotal role in enhancing inflammation (Littman and Rudensky, 2010). The DLNs and splenocytes from both Wt and miR-155−/− mice were examined for the presence of IL-17 (T_{H}17) or IFNγ (T_{H}1) producing CD4+ T cells during EAE. On day 25 post-immunization with MOG₃₅₋₅₅, miR-155−/− mice had substantially diminished levels of T_{H}17 cells in both their DLNs and spleens compared to Wt mice (FIGS. 2A and 2B). Moderately reduced levels of IFNγ producing T_{H}1 CD4+ cells were also found in the spleens but not DLNs of MOG₃₅₋₅₅ immunized mice in the absence of miR-155 (FIGS. 2A and 2B). The total numbers of these inflammatory T cell populations in the spleen and DLNs was also similarly reduced in miR-155−/− versus miR-155+/+ mice 25 days after immunization with MOG₃₅₋₅₅ (FIG. 9).

The in vitro recall response to the MOG₃₅₋₅₅ peptide by Wt and miR-155−/− CD4+ T cells from the spleens of EAE mice was also assessed. CFSE-labeled splenocytes from day 25 EAE mice were restimulated in vitro with 20 μg/ml of MOG₃₅₋₅₅ or cultured in medium alone for 72 hours followed by FACS analysis to determine the extent of CD4+ proliferation as determined by dilution of CFSE. We found that Wt CD4+ T cells underwent cell divisions following exposure to MOG₃₅₋₅₅, while miR-155−/− CD4+ T cells had a substantially reduced proliferative response to the same peptide (FIG. 2C). In parallel, ³[H] thymidine incorporation assays using total splenocytes produced similar differences (FIG. 2D). Tissue culture supernatants from these experiments were assayed for IL-17A and IFNγ production by ELISA in response to MOG₃₅₋₅₅ stimulation. MOG₃₅₋₅₅ stimulated miR-155−/− splenocytes showed minimal production of both of the assessed cytokines compared to Wt splenocytes further demonstrating a defective CD4+ T cell driven recall response to antigen (FIG. 2E).

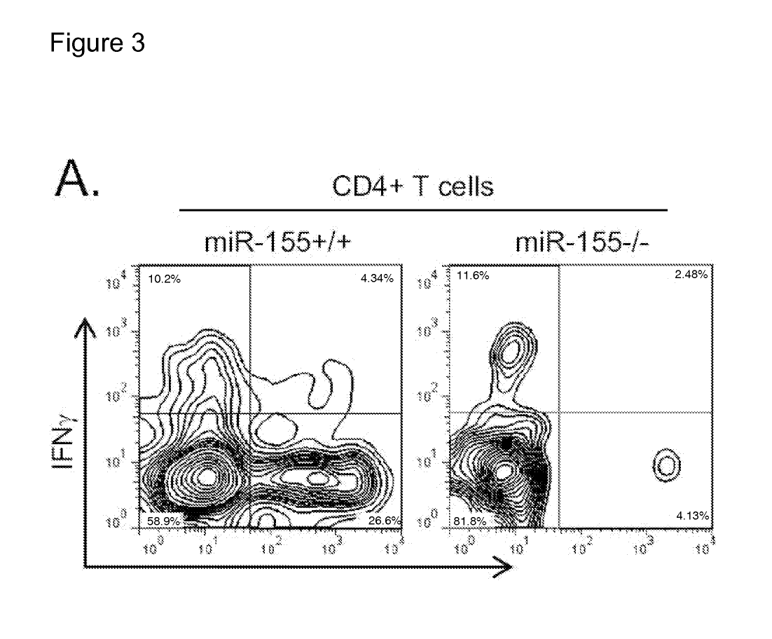

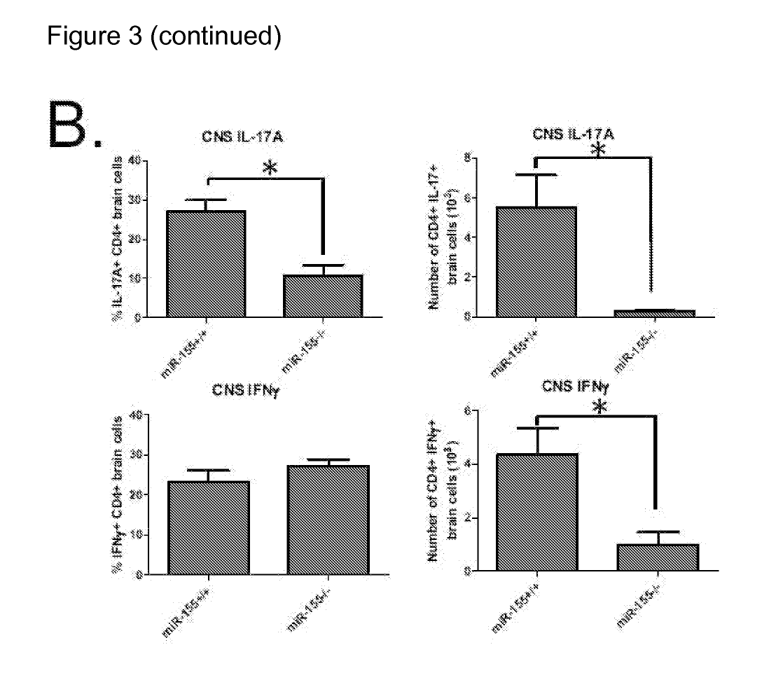

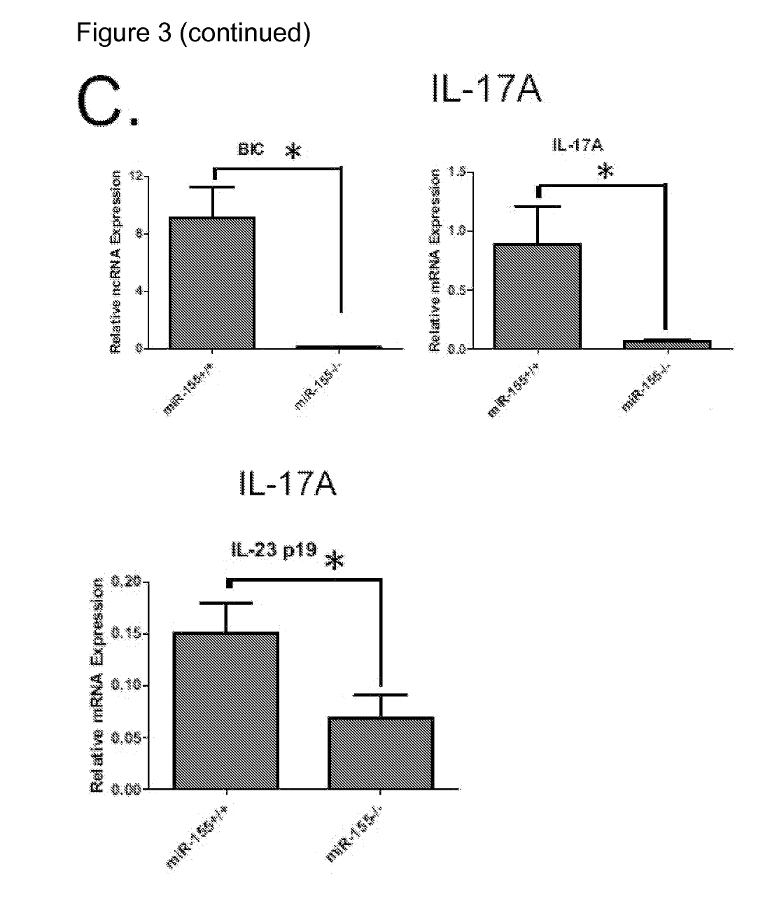

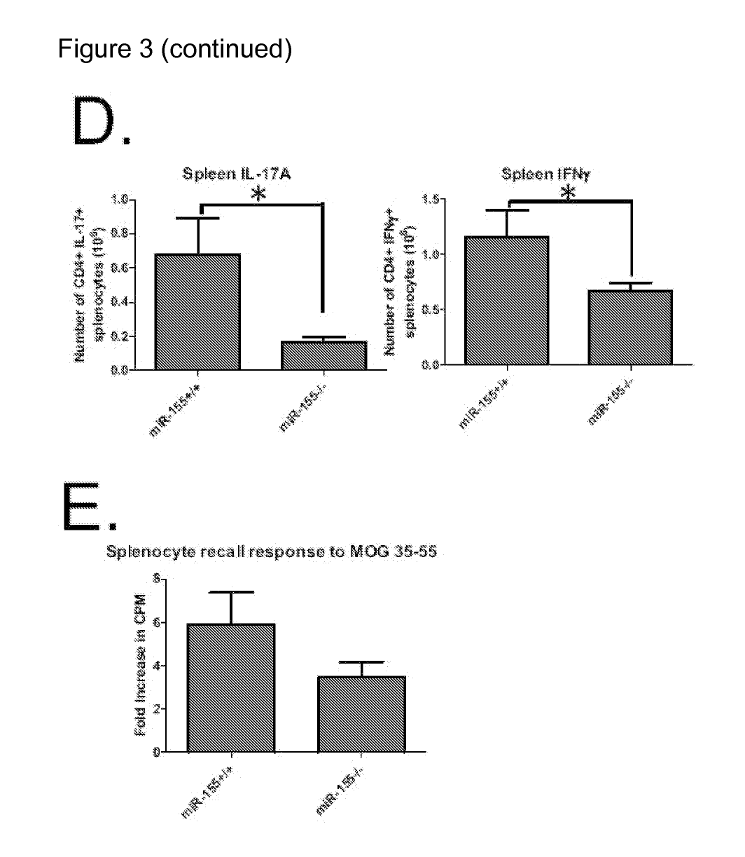

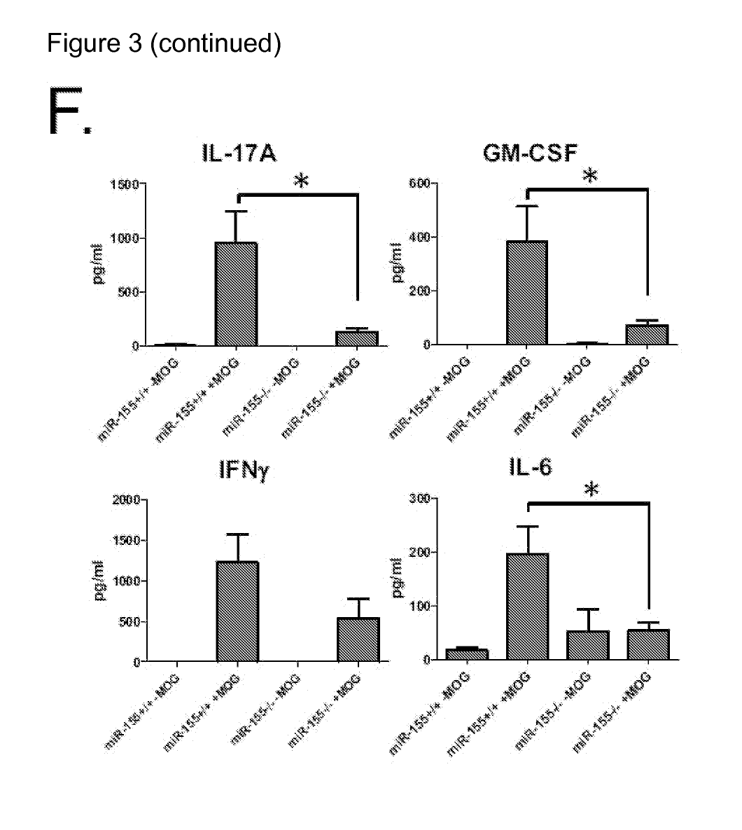

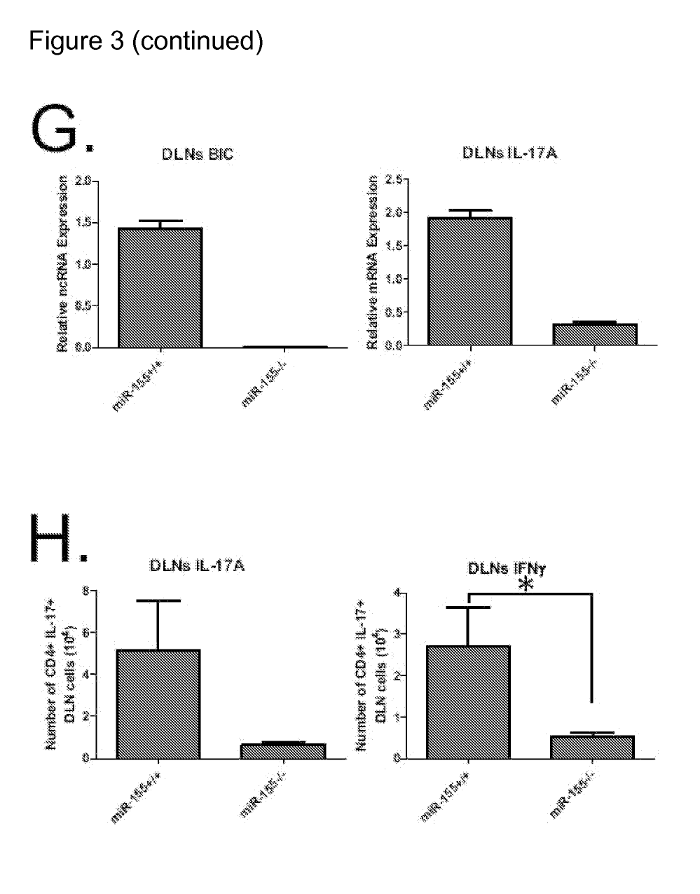

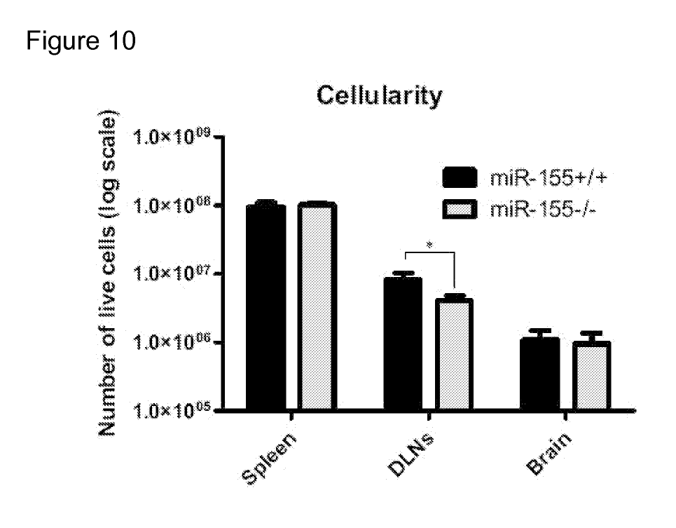

A defect in inflammatory T cell development was also detected during the initial onset, or induction phase, of EAE in mice lacking miR-155. Mice were harvested on day 13 post immunization with MOG₃₅₋₅₅ and the DLNs from miR-155−/− mice had reduced numbers of live cells compared to Wt controls, while the brains (CNS) and spleens from the two groups had similar total cell numbers (FIG. 10). Inflammatory T cell development in the brains, DLNs and spleens was next assessed. miR-155−/− mice had substantial reductions in both the absolute numbers of T_{H}17 cells and the percentage of T_{H}17 cells among total CD4+ T cells in their brains compared to miR-155+/+ control mice (FIGS. 3A and 3B). IFNγ producing T_{H}1 cells were present at lower absolute numbers in the brains of miR-155−/− mice, while the proportion of T_{H}1 T cells among total CD4+ T cells was equivalent between miR-155−/− and miR-155+/+ brains (FIG. 3B). BIC (the ncRNA that gives rise to miR-155) expression was detected by qPCR in miR-155+/+ but not miR-155−/− splenocytes, and deficiencies in both IL-17A and IL-23 p19 mRNAs were also observed (FIG. 3C). Intracellular staining revealed diminished numbers of T_{H}17 and T_{H}1 CD4+ T cells in miR-155−/− spleens (FIG. 3D). The recall response to MOG₃₅₋₅₅ was also tested using splenocytes from day 13 EAE mice. miR-155−/− splenocytes exhibited diminished proliferation during this assay, although this was not statistically significant (FIG. 3E). Defective production of IL-17A, IFNγ, IL-6 and GM-CSF by MOG₃₅₋₅₅ restimulated miR-155−/− encephalitogenic splenocytes was evident (FIG. 3F). Similar deficiencies in T_{H}17 and T_{H}1 cells were also observed in the DLNs from miR-155−/− mice at this same timepoint (FIGS. 3G and 3H). These data indicate that the development of inflammatory T cells in miR-155−/− mice is defective during the early, induction phase of EAE.

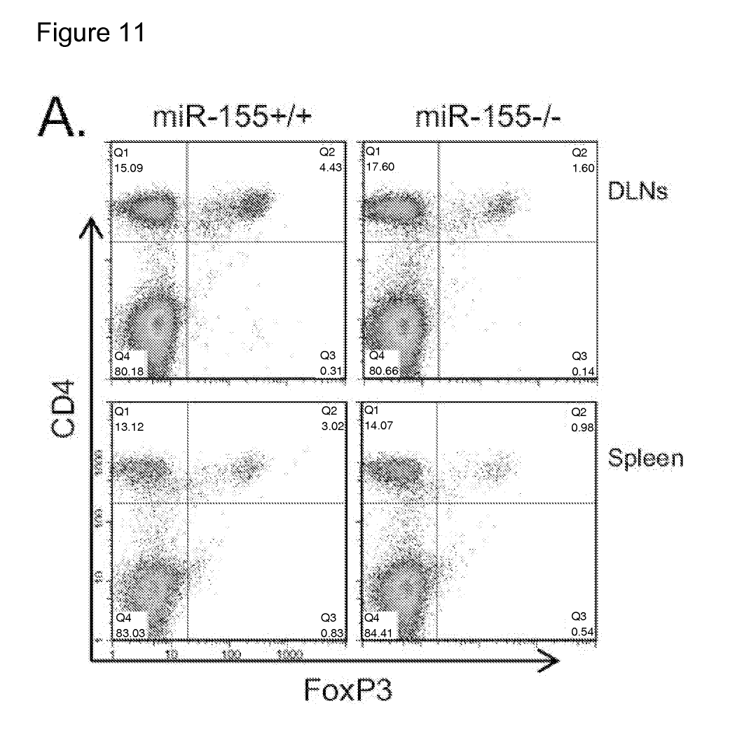

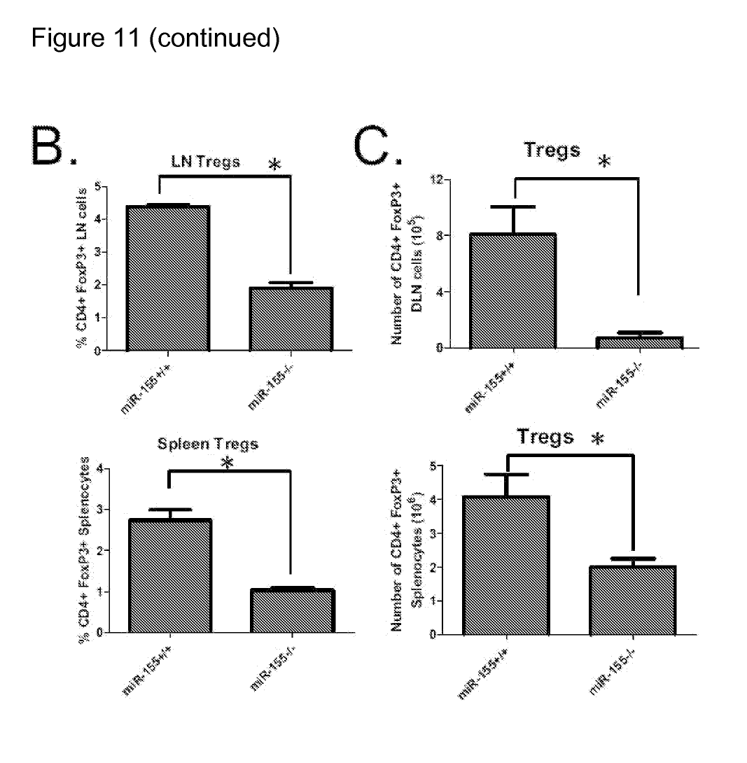

Lower Treg levels were observed in both the DLNs and spleens of miR-155−/− mice compared to Wt controls during EAE (FIG. 11). However, miR-155−/− Tregs are not functionally defective compared to Wt Tregs on a per cell basis. Thus, the reduced EAE inflammation in miR-155−/− mice seems unlikely to be related to the Treg population in these animals.

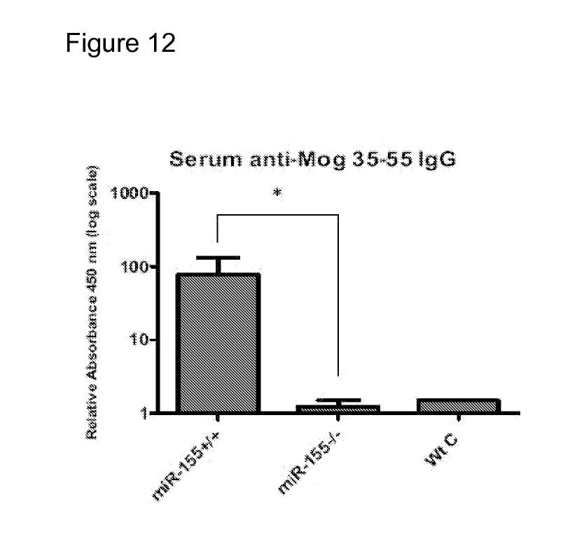

Reduced titers of anti-MOG₃₅₋₅₅IgG antibodies were also seen in miR-155−/− mice during EAE (FIG. 12). Since it has been reported that B cells, and therefore antibodies, are dispensable specifically for MOG₃₅₋₅₅ drive EAE, it is likely that the observed antibody deficit does not account for the reduced disease severity seen in miR-155−/− mice.

mIR-155−/− Mice Have Reduced Foot Pad Inflammation During DTH

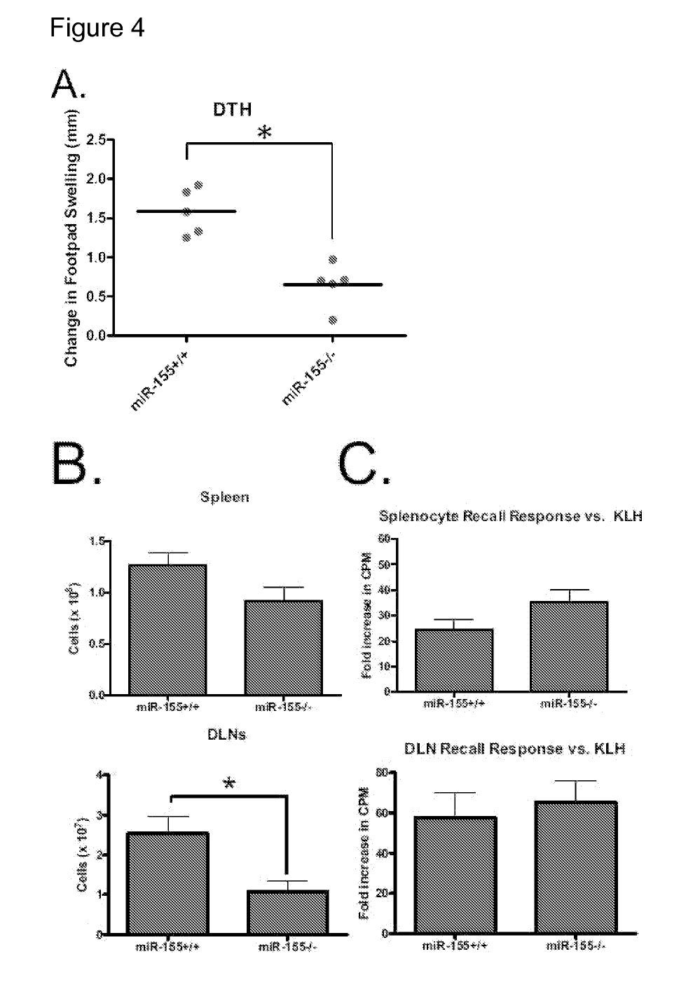

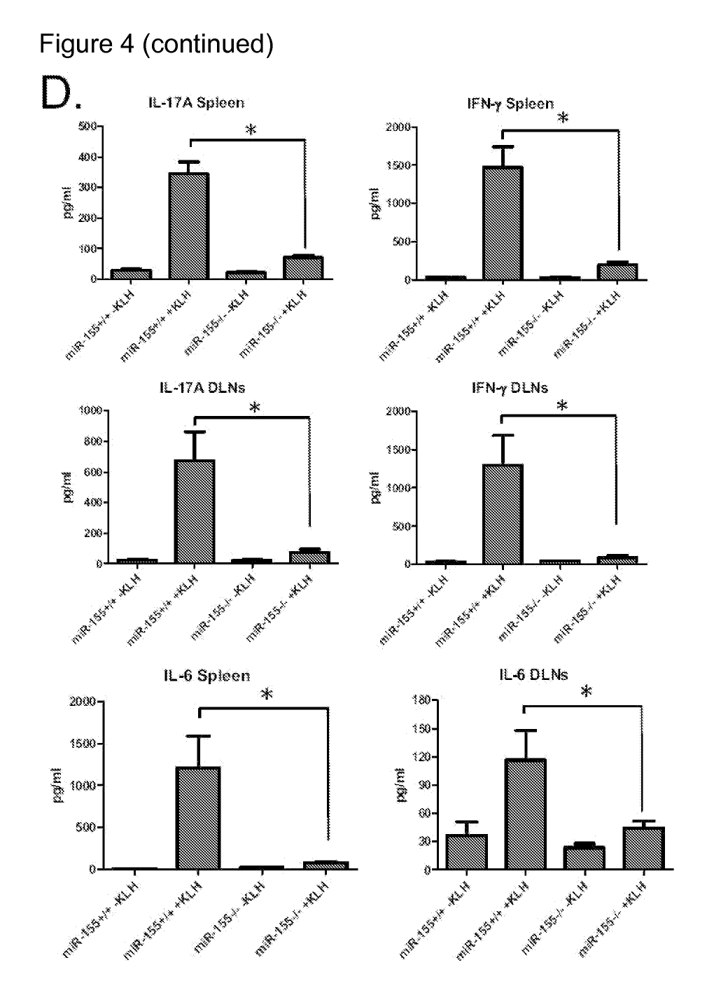

Another T_{H}17 dependent model of inflammation was used to assess whether miR-155−/− mice have a general deficit in mediating inflammatory responses to specific antigens (Ghilardi et al., 2004). Wt and miR-155−/− mice were immunized with Keyhole Limpet Hemocyanin (KLH) in CFA, and 8 days later challenged in one footpad with KLH and in the contralateral footpad with saline. After 48 hours footpad thickness was measured to assess the DTH response. As expected, Wt mice had a substantial increase in footpad inflammation following KLH administration compared to saline alone (FIG. 4A). In contrast, miR-155−/− mice exhibited reduced levels of swelling in response to KLH as compared to Wt mice (FIG. 4A). Splenocytes and DLNs were also harvested on day 10, and miR-155−/− DLNs had significantly fewer total cells than Wt controls, consistent with a blunted inflammatory response (FIG. 4B). Cells from both organs were restimulated with KLH for 3 days. While DLN cell and splenocyte proliferative differences were not observed in response to stimulation with KLH (FIG. 4C), substantial reductions in IL-17A, IFNγ and IL-6 production were seen in miR-155−/− versus Wt splenocytes and DLN cells during recall responses (FIG. 4D). These data reveal a general role for miR-155 in mediating antigen and tissue specific inflammation and point to a consistent defect in inflammatory T cell production.

A T Cell Intrinsic Role for miR-155 in the Development of Inflammatory T Cells During EAE

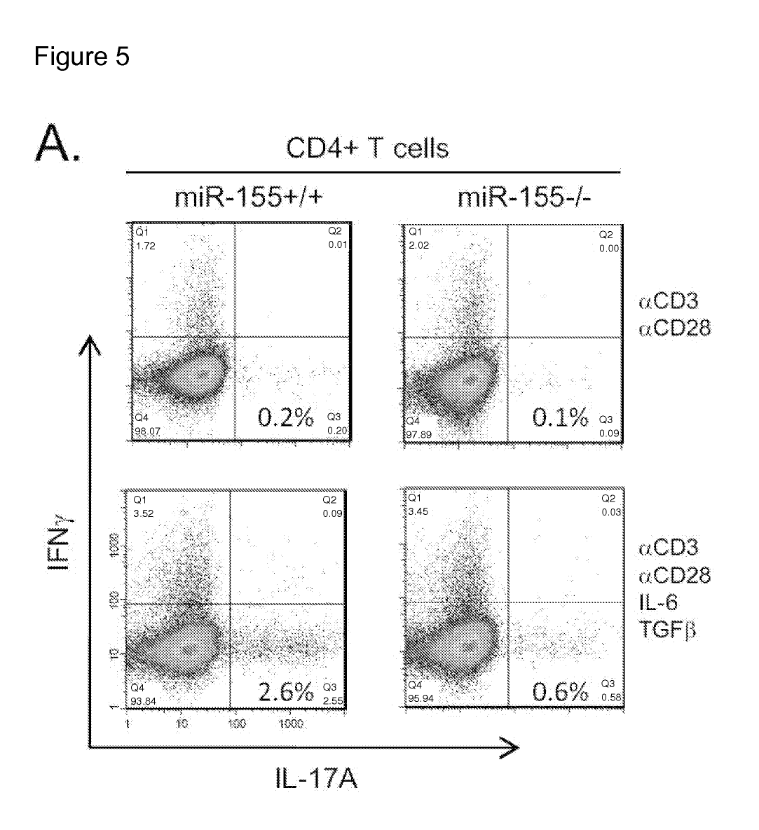

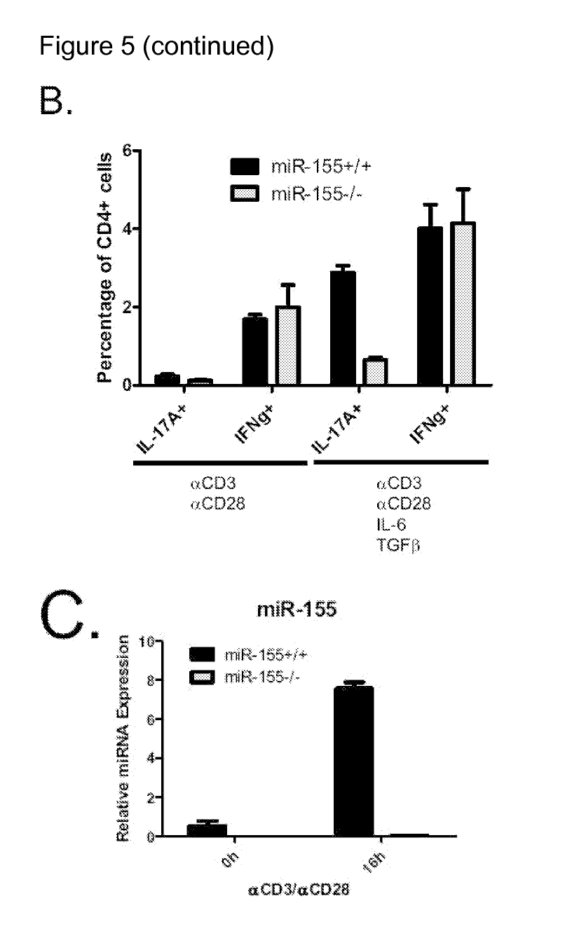

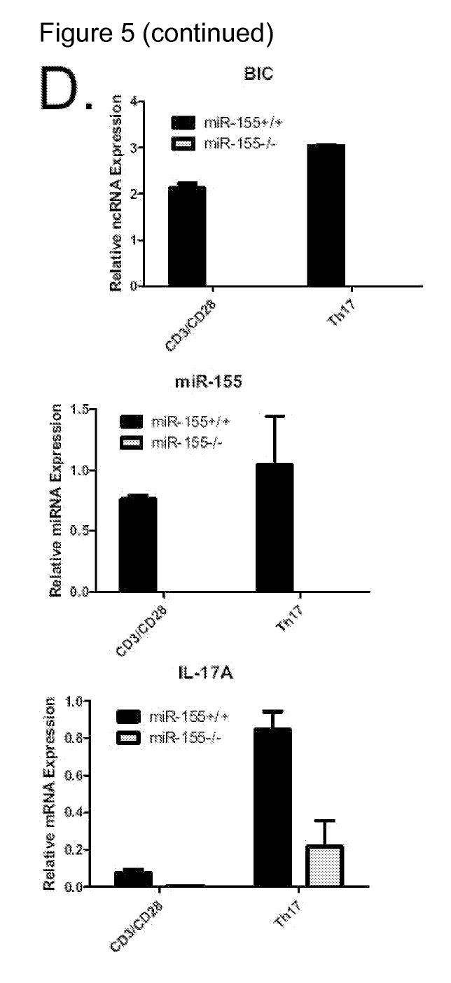

Inflammatory T cells receive and coordinate the signals provided by specific inflammatory cytokines that mediate their development. Thus, the involvement of miR-155 expression by T cells in their ability to be skewed towards the T_{H}17 lineage in vitro was tested. CD4+ splenic T cells were isolated from miR-155+/+ and miR-155−/− mice and cultured in the presence of αCD3 and αCD28 antibodies with and without the addition of the T_{H}17 skewing factors IL-6 and TGFβ. Following four days of culture, we found that miR-155−/− CD4+ T cells were defective in their ability to produce T_{H}17 cells compared to Wt controls as assayed by intracellular staining of IL-17A (FIGS. 5A and 5B). The same cell populations and culture conditions produced similar levels of IFNγ+T_{H}1 cells despite a miR-155 deficiency (FIGS. 5A and 5B). Upregulation of miR-155 in activated CD4+ T cells (FIG. 5C) was also observed, and expression of BIC and miR-155 in CD4+ T cells grown in conditions that promote T_{H}17 development was detected (FIG. 5D). Reduced expression of IL-17A mRNA was measured in miR-155−/− compared to Wt CD4+ T cells under T_{H}17 skewing conditions (FIG. 5D). These results reveal a T cell intrinsic role for miR-155 in promoting the development of T_{H}17 cells.

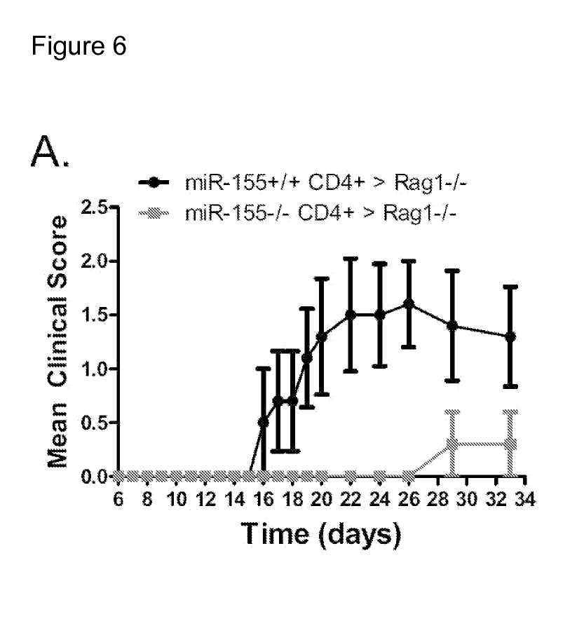

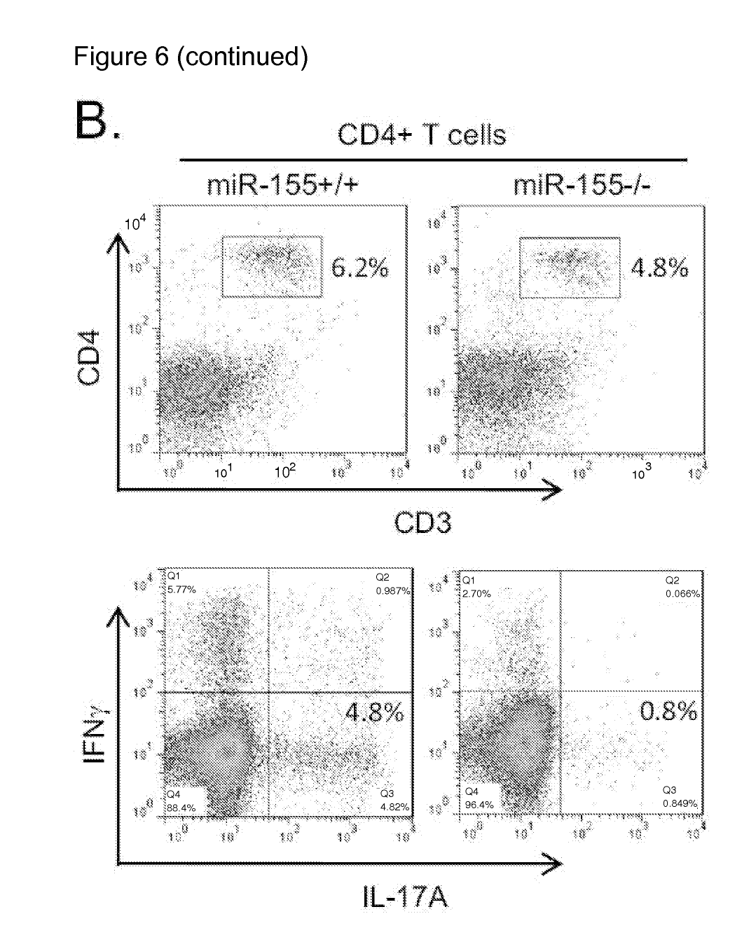

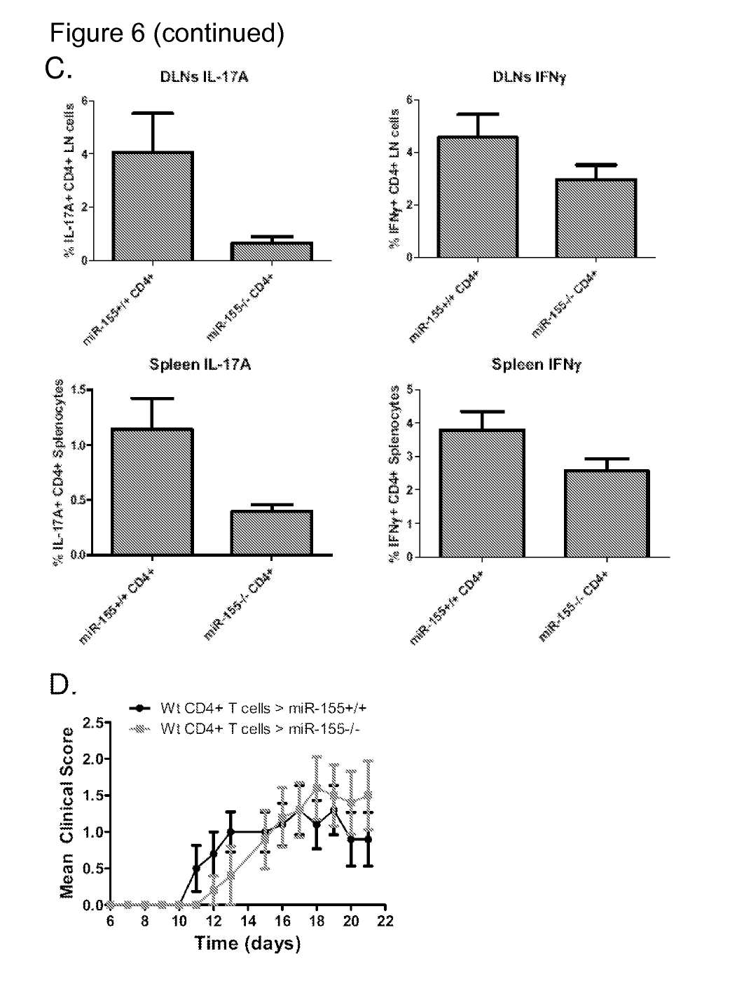

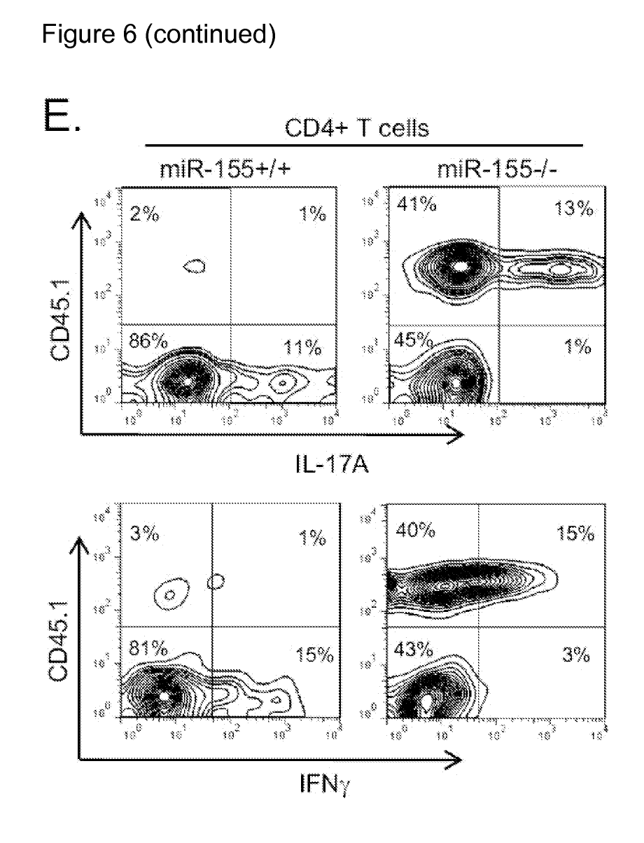

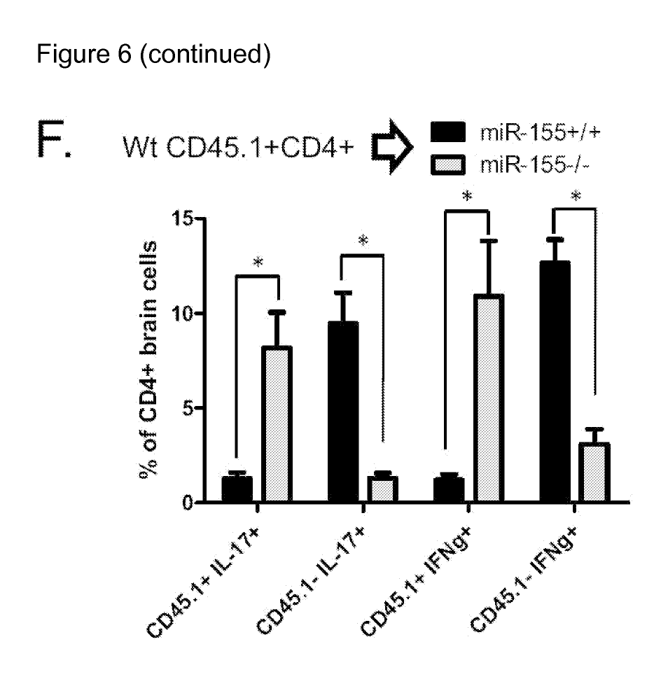

To test whether miR-155 plays a T cell intrinsic role in driving inflammatory T cell development during EAE, 6×10⁶ purified naïve miR-155+/+ or miR-155−/− CD4+ T cells were adoptively transferred into Rag1−/− recipients and EAE was induced 24 hours later. Mice receiving miR-155+/+ CD4+ T cells had a substantially more severe and accelerated disease course compared to mice receiving miR-155−/− CD4+ T cells (FIG. 6A). Reduced percentages of T_{H}17, and to a lesser extent T_{H}1, CD4+ T cells was observed in the spleens and DLNs from mice that received miR-155−/− CD4+ T cells compared to those engrafted with miR-155+/+ CD4+ T cells (FIGS. 6B and 6C).

To examine whether miR-155 expression specifically in CD4+ T cells could restore disease severity in miR-155−/− mice, 10⁷ naïve CD45.1+ CD4+ T cells were adoptively transferred into both Wt and miR-155−/− recipients and EAE was induced 24 hours later. Although miR-155+/+ mice began to show clinical symptoms a few days before miR-155−/− mice, both groups exhibited similar disease scores for most of the time course (FIG. 6D). The brain was harvested on day 23 post-immunization, and the development of T_{H}17 and T_{H}1 cells was determined by intracellular staining for IL17A and IFNγ, respectively. While both mouse groups had roughly equivalent percentages of both Th17 and Th1 cells among the total CD4+ T cells in the brain, these cellular subsets were comprised predominately of the adoptively transferred CD45.1+ CD4+ Wt T cells in the CNS of miR-155−/− mice. This bias occurred despite roughly similar levels of both Wt and miR-155−/− CD4+ T cells being present in the brains of miR-155−/− EAE mice (FIGS. 6E and 6F). Conversely, these same inflammatory T cell populations were comprised largely of endogenous origin (CD45.1-CD4+ miR-155+/+ T cells) in the CNS of miR-155+/+ mice (FIGS. 6E and 6F). These data demonstrate that miR-155 expression by CD4+ T cells is critical for the proper development of inflammatory T cells subsets in the CNS and that this accounts for a majority of miR-155's contribution to EAE.

miR-155 Expression in LPS-Activated, GM-CSF-Derived Myeloid Dendritic Cells is Necessary for Proper Production of Th17 Relevant Inflammatory Cytokines

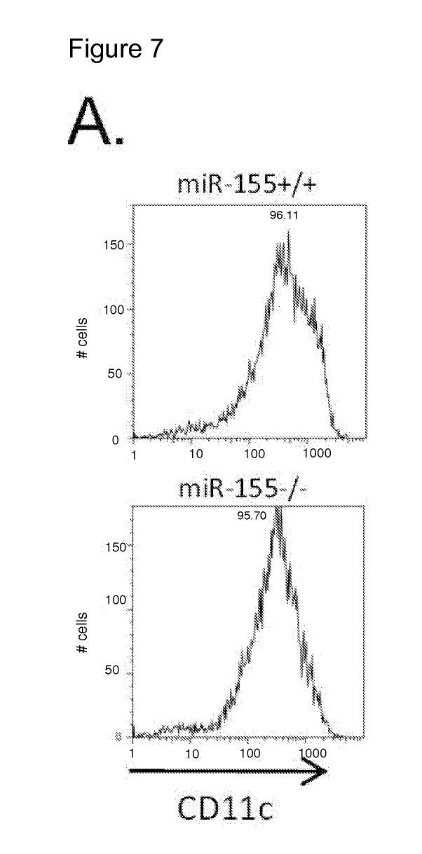

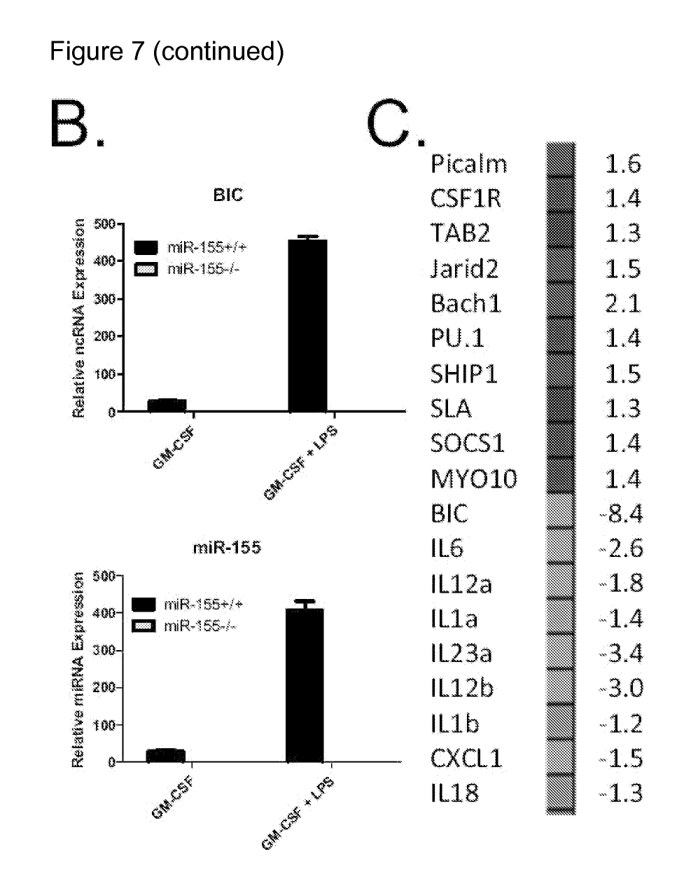

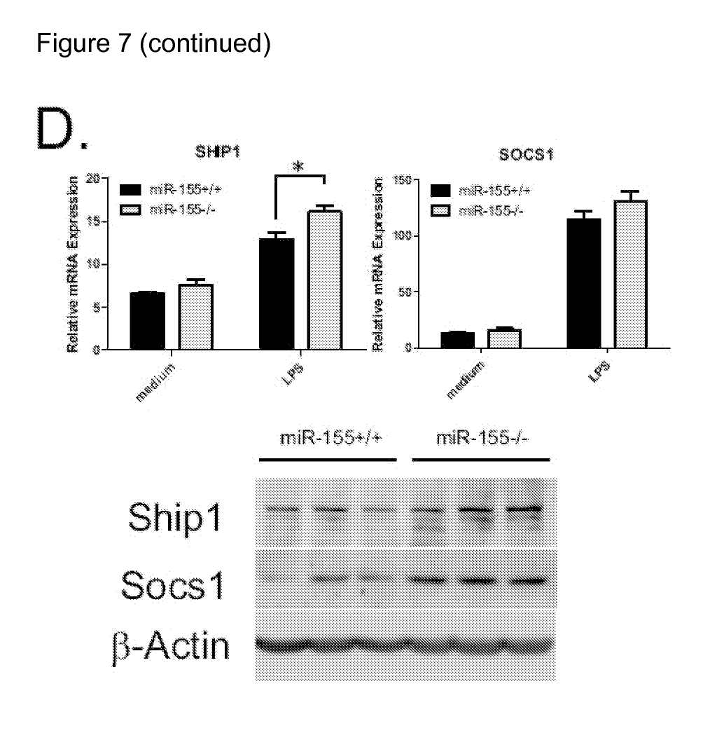

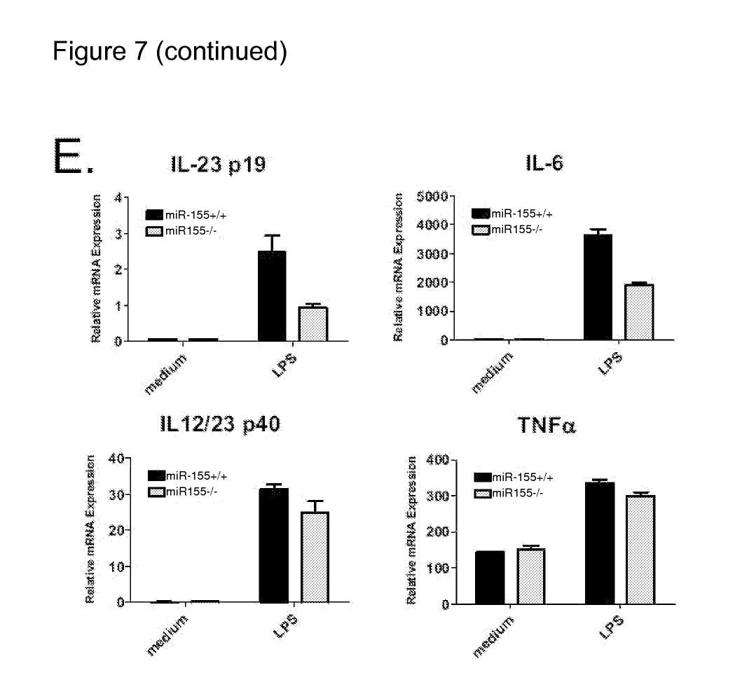

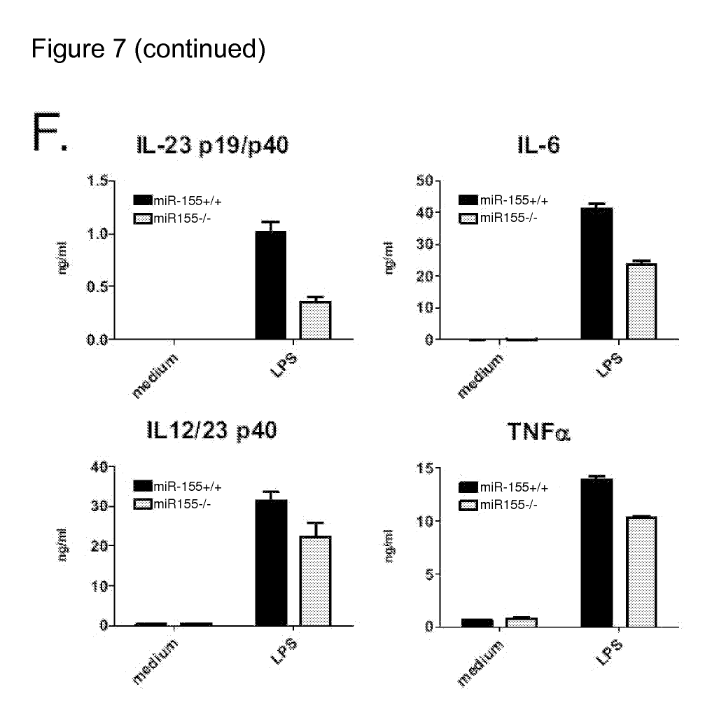

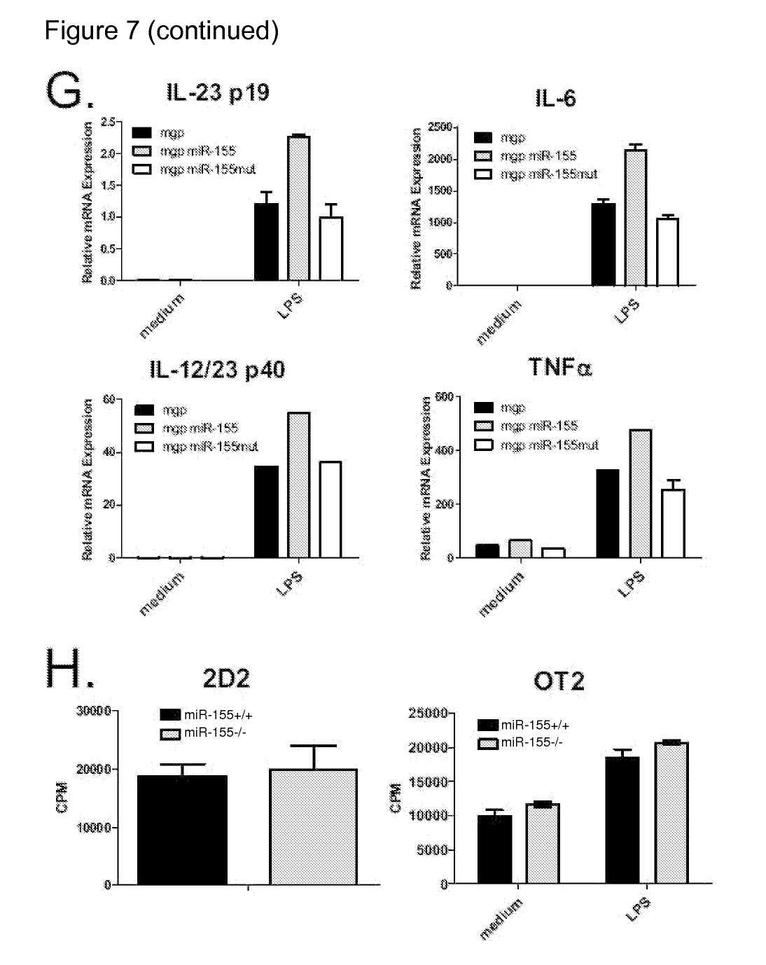

Due to the lag in EAE phenotype “rescue” by Wt CD4+ T cells following their administration to miR-155−/− mice, the function of miR-155 in other immune cell types to promote inflammatory T cell development was investigated. For encephalitogenic T_{H}17 cells to develop they must receive signals from relevant inflammatory cytokines, such as IL-6 and IL-23, which are produced by GM-CSF-derived DCs. Therefore, the impact of miR-155 expression on these or other proinflammatory factors produced by DCs was investigated. Bone marrow from Wt or miR-155−/− mice was differentiated into CD11c+ myeloid DCs using rGM-CSF (FIG. 7A). Of note, GM-CSF-derived DCs were found to express high basal levels of miR-155 compared to myeloid cells derived using M-CSF or IL-3. Following DC activation by LPS, a further increase of miR-155 expression was observed indicating a role for miR-155 in activated GM-CSF derived DCs (FIG. 7B).

To examine the impact of miR-155 on the gene expression profile of activated DCs, total RNA was collected from purified Wt or miR-155−/− DCs after 20 hours of LPS treatment and subjected to a microarray analysis. Several targets of miR-155 were expressed at higher levels in miR-155−/− vs. Wt control DCs (FIG. 7C). Among these, SHIP1 and SOCS1 have been shown to be directly targeted by miR-155 (Androulidaki et al. (2009). The kinase Akt1 controls macrophage response to lipopolysaccharide by regulating microRNAs. Immunity 31, 220-231; Lu et al. (2009). Foxp3-dependent microRNA155 confers competitive fitness to regulatory T cells by targeting SOCS1 protein. Immunity 30, 80-91; O'Connell et al. (2009). Inositol phosphatase SHIP1 is a primary target of miR-155. Proc Natl Acad Sci USA 106, 7113-7118), and to function by negatively regulating cytokine production in DCs (An et al. (2005). Src homology 2 domain-containing inositol-5-phosphatase 1 (SHIP1) negatively regulates TLR4-mediated LPS response primarily through a phosphatase activity- and PI-3K-independent mechanism. Blood 105, 4685-4692; Shen et al. (2004). Silencing of SOCS1 enhances antigen presentation by dendritic cells and antigen-specific anti-tumor immunity. Nat Biotechnol 22, 1546-1553). Their elevated expression in miR-155−/− DCs was confirmed using quantitative PCR (qPCR) and by Western blotting (FIG. 7D). Consistent with the elevated expression of these negative regulators, decreased expression of several inflammatory cytokine genes including IL-6, IL-23 p19 and IL12/23 p40 was observed in miR-155−/− DCs (FIG. 5C). These results were confirmed by qPCR and ELISA, which also detected a subtle decrease in TNF-α production (FIGS. 7E and 7F). To further corroborate these findings, miR-155 was overexpressed in GM-CSF derived DCs using a retroviral vector described previously (O'Connell et al. (2009). Inositol phosphatase SHIP1 is a primary target of miR-155. Proc Natl Acad Sci USA 106, 7113-7118), and higher levels of IL-6, IL-23 p19, IL12/23 p40, and TNF-α mRNA expression were observed following LPS treatment (FIG. 7G). miR-155−/− DCs were also tested for their ability to induce CD4+ T cell proliferation following presentation of cognate antigens. Both miR-155+/+ and miR155−/− DCs induced equivalent proliferation of 2D2 or OT2 CD4+ T cells which recognize MOG₃₅₋₅₅ or an ovalbumin peptide, respectively. Taken together, these experiments demonstrate that miR-155 promotes DC expression of specific cytokines required for inflammatory T cell development.

### Experimental Procedures for Examples 1-5

Mice. All experiments were approved by the Caltech Institutional Animal Care and Use Committee (IACUC). miR-155+/+ and miR-155−/− mice on a C57BL6 genetic background were obtained from Martin Turner at the Sanger Institute, backcrossed to an F9 C57BL6 generation, and subsequently maintained in the Caltech animal facility. Rag1−/−, CD45.1+, OT2 and 2D2 mice, all on a C57BL6 genetic background, were obtained from the Jackson Laboratories.

Mouse models of EAE and DTH. For induction of EAE, mice were injected s.c. into the base of the tail with a volume of 200 μl containing 100 μg/ml MOG₃₅₋₅₅ peptide (GenScript) emulsified in complete Freund's adjuvant (CFA). CFA consisted of incomplete Freund's adjuvant with 4 mg/ml dessicated Mycobacterium Tuberculosis H37 Ra (Difco). Mice were also injected i.p. with 200 ng of pertussis toxin on days 0 and 2, and clinical symptoms scored regularly according to the following criteria: 0—No symptoms, 0.5—Partially limp tail, 1—Completely limp tail, 1.5—Impaired righting reflex, 2—Hindlimb paresis, 2.5—Hindlimb paralysis, 3—Forelimb weakness, 4—Complete paralysis, 5—Death. For some experiments, Wt or miR-155−/− CD4+ T cells were adoptively transferred i.v. into Rag1−/− recipients 24 hours before inducing EAE, or Wt CD45.1+ CD4+ T cells were transferred i.v. into miR-155+/+ or miR-155−/− recipients 24 hours before induction of EAE. For induction of DTH responses, Keyhole limpet hemocyanin (KLH) was purchased from Calbiochem. Mice were immunized s.c. at the base of the tail with 100 μg KLH in 200 μl CFA. To assess DTH, all mice involved in the studies were given 50 μg KLH in 50 μl PBS intradermally into the left foot pad and 50 μl PBS alone in the right foot pad 8 days after the immunization. Two days later, foot pad swelling was measured with a micrometer and recorded as the difference between the left and right foot pad.

Cell culture and reagents. Myeloid DCs were derived from WT or miR-155−/− RBC-depleted bone marrow using rGM-CSF (Ebioscience) at a concentration of 20 ng/ml in complete RPMI (supplemented with 10% FBS, 100 units/ml penicillin, 100 units/ml streptomycin, 50 μM beta-marcaptoethanol). Cells were cultured at 5% CO₂ and 37° C. in a humidified incubator. DCs were stimulated with E. coli LPS (Sigma) at a concentration of 100 ng/ml. For T_{H}17 skewing, CD4+ splenocytes were cultured in complete RPMI, plate bound αCD3 antibodies, and soluble αCD28 antibodies (2 μg/ml), IL-6 (50 ng/ml) and TGFβ (2 ng/ml) (all from Biolegend) for 96 hours. Splenocytes or DLN cells were also cultured in complete RPMI during restimulation with relevant antigens. The MOG₃₅₋₅₅ peptide was synthesized by Genscript. KLH was obtained from Calbiochem. For CFSE experiments, 25×10⁶ splenocytes were labeled in 5 μM CFSE for 10 minutes at 37° C., and subsequently washed 2 times with PBS before culturing. Cellular proliferation was also assayed by pulsing cells with ³[H] thymidine (1 μCi/well) for the final 18 hrs. For co-culture assays, Wt or miR-155+/+ DCs were pulsed with the MOG₃₅₋₅₅ peptide or Ovalbumin and used to activated purified 2D2 or OT2 CD4+ T cells, respectively, at a 1:10 ratio. For adoptive transfer experiments, splenocytes were obtained from day 12 EAE Wt mice and cultured in complete RPMI supplemented with 20 μg/ml MOG₃₅₋₅₅ and 20 ng/ml IL-12 for 2 days before cells were washed and injected intravenously.

Intracellular staining and FACS. To detect intracellular expression of IL-17A, IFNγ or FoxP3 in CD4+ splenocytes, DLNs, or brains cells (purified using Percoll) were first treated with 750 ng/ml ionomycin and 50 ng/ml PMA (Calbiochem) in the presence of 0.5 μl of GolgiPlug (BD biosciences) for 4-5 hrs at 37° C. Cells were subsequently surface stained using αCD4+ antibodies and then permeabilized and fixed in 100 μL of eBioscience Perm/Fix solution overnight at 4° C. Cells were washed once in perm wash buffer (eBioscience) and then stained with 0.3 μg of fluorophore-conjugated anti-IL-17A, IFNγ or FoxP3 (eBioscience) for 20 minutes at 4° C. Fluorophore-conjugated monoclonal antibodies specific to CD11b (Mac1), CD3ε or B220 (eBioscience) were used to stain RBC-lysed splenocytes or DLN cells. Antibodies recognizing CD11c (eBioscience) were also used to stain in vitro derived DCs. After washing, stained cells were assayed using a BD FACSCalibur flow cytometer and results further processed using FlowJo.

Microarray and qPCR. Total RNA was isolated from MACS sorted, LPS activated CD11c+ myeloid DCs derived from Wt or miR-155−/− BM using Trizol (Invitrogen) per manufacturer's instructions. Global mRNA expression levels were next assayed using the Affymetrix total mouse genome array V 2.0 as described previously (O'Connell et al., 2008), and the data was analyzed further using Rosetta Resolver software. Sybrgreen-based quantitative realtime PCR (qPCR) was conducted using the 7300 Realtime PCR system (Applied Biosystems, Foster City, Calif.) to assay BIC, SHIP1, SOCS1, IL-17A, IL-6, IL-23 p19, IL-12/23 p40, TNFα and L32 mRNA levels using gene specific primers (sequences available upon request). Mature miR-155 and sno202 RNA levels were assayed using specific Taqman probes from Applied Biosystems. For all experiments, mRNA was normalized to L32 and miRNA to sno202.

ELISAs. To detect protein expression of GM-CSF, IL-6, IL-17A, IFNγ, IL-23 p19/p40, IL-12/23 p40 and TNF-α, ELISAs were performed using cytokine specific kits form eBioscience and carried out according to the manufacturer's instructions. Serum IgG antibodies against MOG₃₅₋₅₅ were assayed by plating serial dilutions of mouse serum on plates coated with MOG₃₅₋₅₅ and specific antibodies detected using biotinylated anti-mouse IgG antibodies and Streptavidin HRP (Southern Biotech).

Western blotting. Cellular extract was size fractionated using SDS-PAGE and Western blotting was performed as described. Specific antibodies were used to detect SHIP1, SOCS1 and βActin.

Retrovirus production and infections. MSCV-based retroviruses expressing murine miR-155 were prepared as described previously (O'Connell et al. (2009). Inositol phosphatase SHIP1 is a primary target of miR-155. Proc Natl Acad Sci USA 106, 7113-7118), and used to spin infect freshly isolated Wt bone marrow. Immediately following, cells were cultured in GM-CSF containing medium until day 7 before LPS stimulation. Cultures were checked for miR-155 overexpression on day 7 using qPCR.

Histological examination of central nervous system tissues. Brains and spinal cords from EAE mice were dissected and fixed in formaldehyde for 48 hours. Tissue sections were next prepared, stained with H&E and visualized with a Nikon Eclipse 50i microscope, and photographed using a Spot® Digital Camera and software. Sections were scored by a pathologist blinded to the genotype of the tissue or the clinical severity of disease according to the following criteria: 0—no sign of infiltrate, 1—perivascular congestion (light), 2—perivascular congestion (heavy), 3—perivascular congestion (heavy) and parenchymal infiltrate, 4—focal meningeal lymphocytosis, 5—extensive sclerosis.

Statistical analysis. Statistical significance was determined by performing a two-tailed t-test. P values <0.05 were considered significant.

### Example

### Treatment of Autoimmune Disorders

This example illustrates the treatment of a subject suffering from an autoimmune disorder, such as multiple sclerosis (MS).

A subject suffering from or at risk of developing an autoimmune disease or disorder is identified. The subject is identified by any means known to those skilled in the art. The subject is administered an effective amount of an miR-155 antagonist, such as an miR-155 antisense compound. A typical daily dose for an miR-155 antagonist might range from about 0.01 μg/kg to about 1 mg/kg of patient body weight or more per day, and can be determined by the skilled artisan based on a number of factors, including but not limited to the nature of the miR-155 antagonist, the route of administration, and the subject's disease state. MS treatment efficacy is evaluated by observing a delay or slowing of the disease progression, amelioration or palliation of the disease state or symptoms, and/or remission.

### Example

### Treatment of Inflammation

This example illustrates the treatment of a subject suffering from T cell mediated inflammation. A subject suffering from or at risk of developing T cell-mediated inflammation is identified. The subject is administered an effective amount of an miR-155 antagonist, such as an miR-155 antisense compound. A typical daily dose for an miR-155 antagonist might range from about 0.01 μg/kg to about 1 mg/kg of patient body weight or more per day and can be readily determined by the skilled artisan based on a number of factors, including but not limited to the nature of the miR-155 antagonist, the route of administration, and the subject's state. Efficacy is evaluated by observing a reduction of inflammation, or a delay or slowing of an increase in the inflammation. In some embodiments, a reduction in proliferation of CD4+ T cells may be measured.

What is claimed is:

1. A method of reducing tissue specific autoimmune inflammation in a subject, comprising:
identifying a subject in need of a reduction in autoimmune inflammation;
administering an antisense miR-155 oligonucleotide to said subject; and
measuring a reduction in autoimmune inflammation.

2. The method of claim 1, wherein administering comprises delivering the antisense miR-155 oligonucleotide to a population of CD4+ T cells.

3. The method of claim 2, wherein delivering the antisense miR-155 oligonucleotide to CD4+ T cells comprises contacting the CD4+ T cells with an expression vector encoding the antisense miR-155 oligonucleotide.

4. The method of claim 2, additionally comprising measuring proliferation of CD4+ T cells.

5. The method of claim 2, wherein the population of CD4+ T cells comprises T_{H}17 and T_{H}1 T cells.

6. The method of claim 1, wherein the antisense miR155 oligonucleotide comprises a nucleic acid sequence complementary to a miR-155 nucleic acid selected from SEQ ID NO: 1, SEQ ID NO: 2, SEQ ID NO: 3, SEQ ID NO: 4 and SEQ ID NO: 5.

7. The method of claim 1, wherein the antisense miR155 oligonucleotide is capable of hybridizing under high stringency conditions to a miR-155 nucleic acid selected from SEQ ID NO: 1, SEQ ID NO: 2, SEQ ID NO: 3, SEQ ID NO: 4, and SEQ ID NO: 5.

8. A method of decreasing CD4+ T cell proliferation comprising:
administering an antisense miR-155 oligonucleotide to CD4+ T cells; and
measuring proliferation of CD4+ T cells.

9. The method of claim 8, wherein administering an antisense miR-155 oligonucleotide comprises administering an antisense miR-155 expression vector to a target cell such that an antisense miR-155 oligonucleotide is expressed in the target cell.

10. The method of claim 8, wherein the CD4+ T cells are T_{H}17 and T_{H}1 T cells.

11. The method of claim 8, wherein administering an antisense miR-155 oligonucleotide to CD4+ T cells comprises delivering the antisense miR-155 oligonucleotide to a hematopoietic stem cell.

12. The method of claim 8, wherein administering an antisense miR-155 oligonucleotide to CD4+ T cells comprises delivering the antisense miR-155 oligonucleotide to a tissue comprising CD4+ T cells.

13. The method of claim 8, wherein the antisense miR155 oligonucleotide comprises a nucleic acid sequence complementary to a miR-155 nucleic acid selected from SEQ ID NO: 1, SEQ ID NO: 2, SEQ ID NO: 3, SEQ ID NO: 4, and SEQ ID NO: 5.

14. A method of decreasing cytokine production by dendritic cells comprising:
inhibiting miR-155 activity in the dendritic cells, wherein the cytokine is selected from the group consisting of IL-23/IL-17, GM-CSF, IL-6, IFNγ and TNF-α.

15. The method of claim 14, wherein miR-155 activity is inhibited in the dendritic cells by delivering an antisense miR-155 oligonucleotide to the dendritic cells.

16. A method of increasing immune response to an infectious agent comprising:
identifying a subject suffering from infection by the infectious agent;
administering an antisense miR-155 oligonucleotide to the subject; and
measuring proliferation of CD4+ T cells in the patient.

17. The method of claim 16, wherein the infectious agent is selected from bacteria and viruses.

18. The method of claim 16, wherein CD4+ T cells are T_{H}17 or T_{H}1 T cells.

19. A method of treating an autoimmune disorder in a patient comprising:
identifying a patient suffering from tissue specific autoimmune inflammation;
administering an antisense miR-155 oligonucleotide to the patient; and
measuring a reduction in tissue specific autoimmune inflammation.

20. The method of claim 19, wherein the antisense miR-155 oligonucleotide is delivered to a tissue comprising CD4+ T cells.

21. The method of claim 19, wherein the antisense miR155 oligonucleotide comprises a nucleic acid sequence complementary to a miR-155 nucleic acid selected from SEQ ID NO: 1, SEQ ID NO: 2, SEQ ID NO: 3, SEQ ID NO: 4, and SEQ ID NO: 5.

22. The method of claim 19, wherein the autoimmune disorder is selected from the group consisting of multiple sclerosis, rheumatoid arthritis, irritable bowel syndrome and psoriasis.

23. A method of reducing development of T_{H}1 and T_{H}17 cells in a tissue undergoing autoimmune inflammation, comprising:
identifying a tissue undergoing autoimmune inflammation;
administering an antisense miR-155 oligonucleotide to said tissue.

24. The method of claim 23, wherein administering the antisense miR-155 oligonucleotide to the tissue comprises contacting the tissue with an expression vector encoding the antisense miR-155 oligonucleotide.

25. The method of claim 23, additionally comprising measuring development of T_{H}1 and T_{H}17 cells in said tissue.
